(19) United States
(12) Patent Application Publication
(10) Pub. No.: US 2007/0122430 A1
(43) Pub. Date: May 31, 2007

(54) Influenza vaccine compositions and methods of use thereof

(75) Inventors: Alexander M. Shneider, Stoughton, MA (US); Peter Ilyinskii, Somerville, MA (US); Galini Thoidis, Boston, MA (US)

Correspondence Address: DLA PIPER US LLP, 4365 EXECUTIVE DRIVE, SUITE 1100, SAN DIEGO, CA, 92121-2133, US

(73) Assignee: Cure Lab, Inc., Canton, MA (US)

(21) Appl. No.: 11/498,320

(22) Filed: Aug. 1, 2006

Related U.S. Application Data
(63) Continuation-in-part of application No. 11/380,554, filed on Apr. 27, 2006.
(60) Provisional application No. 60/704,586, filed on Aug. 1, 2005.

(51) Int. Cl. A61K 39/395; A61K 39/12

(52) U.S. Cl. 424/204.1; 424/130.1

(57) ABSTRACT

Compositions of anti-influenza vaccine containing nucleic acids encoding influenza proteins NP, M1 and NS-1 and methods of inducing a protective immune response using these compositions. Also included is the enhancement of antigenic presentation or increasing immunogenicity of an influenza NP, M1 and/or NS-1 polypeptide by modifying the three dimensional structure of the polypeptide.

This application claims priority under 35 U.S.C. §119(e) to U.S. patent application Ser. No. 60/704,586, filed Aug. 1, 2005, and to U.S. patent application Ser. No. 11/380,554, filed Apr. 27, 2006, the entire content of which is incorporated herein by reference.

### FIELD OF THE INVENTION

The present invention relates generally to influenza vaccine compositions, methods of producing such compositions, and methods of use thereof to treat or protect against influenza infection.

### BACKGROUND OF THE INVENTION

Influenza is a contagious respiratory illness caused by orthomyxoviridae, spherical, enveloped viruses, able to attach to cell surface receptors. Influenza regularly affects the world in seasonal epidemics, usually starting between November and March in the Northern Hemisphere and between April and September in the Southern Hemisphere. These epidemics impose a considerable economic burden in the form of health care costs and lost productivity. Each year, approximately 5-15% of the world's population contracts influenza resulting in 3-5 million cases of severe illness. Not only are large numbers of people affected, influenza can cause life threatening complications in the elderly, pregnant women, newborns, and people with certain chronic medical conditions. While usually considered a self-limiting disease, influenza is in fact associated with considerable morbidity and mortality worldwide. Currently, adults over 65 years account for approximately 90% of influenza-related deaths. Globally, an estimated 250,000 to 500,000 people die annually from influenza-associated complications.

Influenza is easily transmitted from person to person. The virus enters the body via the upper respiratory tract with the most significant pathology occurring in the lower respiratory tract. Infection spreads quickly across the population with crowded environments such as schools especially favoring its rapid transmission. The U.S. Centers for Disease Control and Prevention (CDC) estimates that in the U.S., 10-20% of the population is infected with the influenza virus each year, that 114,000 are hospitalized for influenza-related complications, and ˜36,000 die annually.

There are three types of influenza virus: A, B, and C, which vary greatly in their epidemiological pattern. Influenza A virus is both best characterized and the most serious threat to public health, capable of inducing massive epidemics or pandemics. This virus is also highly variable antigenically. Two virus encoded proteins, hemagglutinin (HA) and neuraminidase (NA) constitute the layer of radial spikes over the viral surface. Both of these proteins are essential for viral infection and pathogenesis.

A vaccine to influenza is one of the most efficacious, safe, nontoxic and economical weapons to prevent disease and to control the spread of the disease. Conventional vaccines are a form of immunoprophylaxis given before disease occurrence to afford immunoprotection by generating a strong host immunological memory against a specific antigen. The primary aim of vaccination is to activate the adaptive specific immune response, primarily to generate B and T lymphocytes against specific antigen(s) associated with the disease or the disease agent.

Currently a flu shot made from inactivated whole virus is generally available and is in widespread use. A better approach is the development of a DNA or protein-based vaccine which would induce a permanent immune response (rather than having to administer it yearly, like the current flu shot), and which does not rely on inactivated viruses and the possible side effects of the use thereof, e.g., apprehensions about using whole virus vaccines in pregnant women and other at-risk groups. Furthermore, the current flu vaccines have a disadvantage in that they are narrowly focused on one specific viral strain.

### SUMMARY OF THE INVENTION

The present invention is directed to new vaccine compositions, methods of producing them, and methods of using these vaccines in preventing and treating influenza. The seminal discovery behind this invention features the use of a combination of DNA molecules coding for influenza nucleoprotein (NP), matrix-1 (M1) protein, and Non-structural-1 (NS-1) protein from influenza virus. In certain embodiments, these influenza proteins are modified by the insertion, deletion or substitution of one or more amino acids in an internal region of the influenza protein. While not wanting to be bound by a particular theory, it is believed that the introduction of these modifications changes the conformation of that protein so that the ubiquitin-proteasome system degrades the protein more efficiently than a protein in the absence of the modification, resulting in more peptides that are generated and that bind more frequently to MHC-I, providing a more effective and long-term T cell response. The modified influenza polypeptides of the invention are capable of undergoing more efficient proteolytic cleavage as compared to wild-type proteins; modified polypeptides are generally degraded to one or more peptides of less than about 50, about 25, about 15, about 10 or about 5 amino acids in length.

One aspect of the invention relates to a vaccine containing a first isolated nucleic acid encoding an influenza nucleoprotein, a second isolated nucleic acid encoding an influenza M1 protein, and a third isolated nucleic acid encoding an influenza NS-1 protein, where the vaccine is capable of inducing a protective immune response in a mammal. In certain embodiments, the mammal is a human, such as a human at risk of infection by an influenza virus. Humans at highest risk of infection include young children (e.g., under 5 years of age), the elderly (e.g., over 65 years of age), health care workers, and immunocompromised individuals, in addition to the general population. The invention also provides a method for inducing an immune response against an influenza virus in a subject, administering to the subject this vaccine vaccine.

In one embodiment of the invention, the influenza nucleoprotein, the M1 protein and the NS1 protein are all wild type proteins. In other aspects of the invention, the proteins are a combination of wild type and modified or non-naturally occurring (e.g., synthetic). Nucleic acid sequences encoding the wild type or modified proteins can be separate open reading frames on separate vectors, or a combination thereof. For example, the three nucleic acid sequences can be separate or combined open reading frames in one or more vectors; three nucleic acid sequences can be on three separate vectors, etc. for any combination of genes and vectors possible using the three nucleic acid sequences encoding wild type or modified proteins (or combinations thereof).

In embodiments of the invention, the influenza nucleoprotein is a modified influenza nucleoprotein (NP) having an amino acid sequence that is non-naturally occurring or synthetic. Typically, the modified NP will have one or more hydrophobic amino acids inserted into the core domain of the NP polypeptide (the core domain includes the entire NP polypeptide except for five amino acids at the N-terminus and five amino acids at the C-terminus). Alternatively, the modified NP will have one or substitutions in the core domain, in which one or more hydrophilic amino acids are substituted for by one or more hydrophobic amino acids. The modified NP is more susceptible to proteolysis as compared to a polypeptide having an amino acid sequence identical to the modified NP except at the modification site(s) and at sites of one or more conservative substitutions. A wild-type NP amino acid sequence is provided by SEQ ID NO: 1. A consensus wild-type NP amino acid sequence generated by multiple sequence alignment is provided by SEQ ID NO: X1.

In other embodiments of the invention the influenza M1 protein is a modified M1 protein having an amino acid sequence that is non-naturally occurring. Typically, the modified M1 protein will have one or more hydrophobic amino acids inserted into the core domain of the M1 polypeptide (the core domain includes the entire M1 polypeptide except for five amino acids at the N-terminus and five amino acids at the C-terminus). Alternatively, the modified M1 will have one or substitutions in the core domain, in which one or more hydrophilic amino acids are substituted for by one or more hydrophobic amino acids. The modified M1 is more susceptible to proteolysis as compared to a polypeptide having an amino acid sequence identical to the modified M1 except at the modification site(s) and at sites of one or more conservative substitutions. A wild-type M1 amino acid sequence is provided by SEQ ID NO: 2. A consensus wild-type M1 amino acid sequence generated by multiple sequence alignment is provided by SEQ ID NO: X2.

In other embodiments of the invention the influenza NS-1 protein is a modified NS-1 protein having an amino acid sequence that is non-naturally occurring. Typically, the modified NS-1 protein will have one or more hydrophobic amino acids inserted into the core domain of the NS-1 polypeptide (the core domain includes the entire NS-1 polypeptide except for five amino acids at the N-terminus and five amino acids at the C-terminus). Alternatively, the modified NS-1 will have one or substitutions in the core domain, in which one or more hydrophilic amino acids are substituted for by one or more hydrophobic amino acids. The modified NS-1 is more susceptible to proteolysis as compared to a polypeptide having an amino acid sequence identical to the modified NS-1 except at the modification site(s) and at sites of one or more conservative substitutions. A wild-type NS-1 amino acid sequence is provided by SEQ ID NO: 3. A consensus wild-type NS-1 amino acid sequence generated by multiple sequence alignment is provided by SEQ ID NO: X3. Additionally, in some embodiments the NS-1 amino acid has decreased interferon stimulatory activity as compared to wild-type NS-1 polypeptides.

In certain embodiments, the vaccine contains one modified protein and two wild type proteins; in other embodiments the vaccine contains two modified proteins and one wild-type protein, and in still other embodiments the vaccine contains three modified proteins.

Generally, the nucleic acids of the vaccine are each present in the form of nucleic acid vectors, e.g., a plasmid vector, a viral vector, e.g., a vaccinia virus vector, adeno-associated virus, VEEV, Sendai-based, NDV-based, an adenovirus vector. In one aspect, the vaccine is formulated in a pharmaceutically-effective carrier. While illustrative examples show the vaccine containing three vectors, one for each protein to be expressed, it is understood that one, two or three vectors can be utilized for the vaccine of the invention with various combination of nucleic acid encoding the proteins (wild-type or modified) in one or more open reading frames.

Another aspect of the invention relates to a vaccine containing a first isolated nucleic acid encoding a modified influenza nucleoprotein, a second isolated nucleic acid encoding a modified influenza M1 protein, and a third isolated nucleic acid encoding a modified influenza NS-1, where the vaccine is capable of inducing an immune response in a mammal. The modified NP, M1 and NS-1 proteins are more susceptible to proteolysis as compared to wild-type NP, M1 and NS-1 polypeptides.

The invention also provides a vaccine capable of inducing a protective immune response in a mammal containing an isolated nucleic acid encoding an influenza nucleoprotein, an isolated nucleic acid encoding an influenza M1 protein, and an isolated nucleic acid encoding an influenza NS-1 protein having the amino acid sequence of the polypeptide of SEQ ID NO: 3 or a conservative substitution thereof except that the NS-1 protein has one or more amino acid substitutions or mutations resulting in the NS-1 protein having decreased interferon stimulatory activity as compared to the polypeptide of SEQ ID NO: 3 or a conservative substation thereof. In embodiments, the vaccine contains a modified influenza nucleoprotein and/or a modified M1 protein.

The invention also provides a vaccine containing a nucleic acid vector that includes a nucleic acid encoding an influenza nucleoprotein, a nucleic acid vector that includes a nucleic acid encoding an influenza M1 protein, and a nucleic acid vector that includes a nucleic acid encoding an influenza NS-1 protein. In embodiments, the influenza NS-1 protein is modified such that it has decreased interferon inhibitory activity as compared a polypeptide of a sequence identical thereto except for the modification. In other embodiments, the vaccine is formulated to be suitable for any means of administration, including intraperitoneal, subcutaneous, nasal, intravenous, oral, topical or transdermal in a vector, e.g., viral vector, DNA vector, or an RNA vector or a liposome.

In another aspect, the vaccine of the present invention contains an isolated influenza nucleoprotein, an isolated influenza M1 protein, and an isolated influenza NS-1 protein. In embodiments, the NP, M1 and/or NS-1 proteins are modified influenza proteins. For example, the NS-1 protein has decreased interferon stimulatory activity as compared to the polypeptide of SEQ ID NO: X3.

In a further aspect, the invention provides an attenuated influenza virus comprising an NS-1 protein having a non-naturally occurring amino acid sequence and having decreased interferon inhibitory activity as compared to wild-type NS-1 polypeptides (e.g. the polypeptide of SEQ ID NO: 3.

The invention also provides a method for formulating a vaccine by combining a pharmaceutically acceptable carrier, an isolated nucleic acid encoding an influenza nucleoprotein, an isolated nucleic acid encoding an influenza M1 protein, and an isolated nucleic acid encoding an influenza NS-1 protein.

The invention also provides a method for formulating a vaccine by combining a pharmaceutically acceptable carrier and an attenuated influenza virus comprising an NS-1 protein having an amino acid sequence of the polypeptide of SEQ ID NO: X3 or a conservative substitution thereof, wherein at least one residue has been deleted or substituted such that the NS-1 protein has decreased interferon stimulatory activity as compared to the polypeptide of SEQ ID NO: X3.

The invention further provides a method of formulating a vaccine by combining a pharmaceutically acceptable carrier, an isolated influenza nucleoprotein, an isolated influenza M1 protein, and an isolated non-naturally occurring influenza NS-1 protein, wherein the influenza NS-1 protein is modified such that the NS-1 protein has decreased interferon inhibitory activity as compared to an unmodified NS-1 protein (e.g., the consensus polypeptide of SEQ ID NO: X3).

The invention also provides a vaccine containing an immunogenic peptide derived from an influenza nucleoprotein (e.g., CTELKLSDY, RRSGAAGAAVK, EDLTFLARSAL, ILRGSVAHK, ELRSRYWAI or SRYWAIRTR), an immunogenic peptide derived from an influenza M1 protein (e.g., SGPLKAEIAQRLEDV, GILGFVFTL, or ASCMGLIY), and an immunogenic peptide derived from an influenza NS-1 protein (e.g., DRLRRDQKS and AIMDKNIIL), wherein the vaccine is capable of inducing an immune response in a mammal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A and 1B are photographs of Coomassie blue-stained SDS-polyacrylamide gels (SDS-PAGE) demonstrating expression of wild-type NP, M1, NS-1 and modified NS-1 in 293T cells. Total cell extract was subjected to SDS-PAGE and Coomassie-stained. MW markers and viral proteins positions are shown. FIG. 1A shows expression of NP, M1 and NS1; FIG. 1B shows expression of NS1, NS1del34 and NP in 293T cells. Faint additional band corresponding to NS1del34 is marked.

FIG. 2 is a photograph of a Western blot analysis of expressed wild-type and modified NS-1 proteins in 293T cells. Expression of pCAGGS-driven wild-type NS1 and its mutants in transfected 293T cells is shown. Total cell extract was subjected to SDS-PAGE, transferred to nitrocellulose and treated with polyclonal guinea pig serum against NS1. Lane 1—NS1 wt, lanes 2, 3—NS1del34/184, lanes 4, 5-NS1del34. Equal protein amounts were used in lanes 2-5 and ⅕ of this amount was used in lane 1. Lanes 2, 3 and 4, 5—1 and 2.7 μg of plasmid DNA were used.

FIG. 3 is a bar graph showing CTL response in animals immunized with a vaccine containing DNA plasmids encoding NP, M1 and NS-1polypeptides. BALB/c mice were injected 3 times at 14-day interval and CTL activity was measured as described in text. Positive control—infection with sublethal dose of A/Aichi/2/68 virus, negative control—placebo immunization.

FIG. 4. is a line graph showing antibody reactivity against whole disrupted influenza virus using successive 2-fold dilutions (i.e., “5” on the x-axis indicates a dilution of 1:32) of sera incubated with antigen, level of antiviral antibodies was expressed as optical density (OD). Two-fold dilutions of sera were incubated with plated antigen (whole-disrupted influenza virus A/PR/8/34), and level of antiviral antibodies was determined as described in text.

FIG. 5A and 5B are line graphs demonstrating body weight recovery in influenza-infected animals vaccinated with combinations of NP-, M1- and NS1-expressing plasmids. FIG. 5A shows challenge with 10 LD₅₀ FIG. 1B shows challenge with 100 LD₅₀.

FIG. 6 demonstrates lung pathology in the NP-, M1- and NS1 DNA-vaccinated and experimentally-infected mice. Lung pathology (day 6 after infection) in the vaccinated and experimentally-infected mice from the same experimental groups described in the legend to FIG. 5. Hemorrhagic inflammation areas are shown by arrows.

FIG. 7 is a line graph demonstrating the protection of mice vaccinated with the combination of NP, M1 and NS1 DNA plasmids from morbidity and mortality after challenge with 5 LD₅₀ of avian mouse-adapted H5N2 influenza virus strain (A/Mallard/Pennsylvania/10218/84).

FIG. 8 is a line graph demonstrating the protection of chickens vaccinated with the combination of NP, M1 and NS1 plasmids from morbidity and mortality after challenge with the lethal doses of avian H5N3 influenza virus strain. Birds unvaccinated and vaccinated with either pNP/pM1 or pNP/pM1/pNS1 were challenged with the lethal doses of H5N3 A/Tern/SA/61 avian influenza virus as described in text.

### DETAILED DESCRIPTION OF THE INVENTION

The invention is based in part on an alternative to classic vaccination wherein the T-cell branch of the immune system is directed to attack a viral entity, e.g., viral particles in the serum. In the present invention, the T-cell branch of immune system is activated to target a cell that has been modified by introducing a nucleic acid encoding an influenza peptide has been inserted. Modified cells present peptides derived from pathogen proteins on their surface in complex with MHC-I proteins. If the number of pathogen-derived peptides presented on the cell surface exceeds a threshold, propagation of a specialized clone of T-cells that specifically recognizes the infected cells is induced, and eliminates infected cells. Multiple mechanisms have evolved in viruses that prevent or reduce T-cell immune response. One critical and ubiquitous mechanism is the acquisition by viral proteins of a structure that prevents their degradation by proteasomes and thus reduces their processing and generation of peptides to be presented on MHC-1. For example, NP-protein (nuclear protein or nucleoprotein) of influenza virus is poorly processed by the cellular proteolytic machinery, leading to its poor presentation on MHC-1 and poor activation of T-cell immune response. Influenza NP has a lower rate of mutation as compared to influenza surface proteins (see, e.g., Lee et al., 2001. Arch. Virol. 146:369-77). Influenza nucleoprotein (Influenza A/Puerto Rico/8/34 strain) contains an H-2Kd-restricted CD8+T cell (T CD8+) epitope spanning amino acid residues 147-155. It has been demonstrated that expression of NP147-155 and NP147-158 in isolation via “minigene”/recombinant vaccinia virus (vac) technology leads to sensitization of target cells for NP-specific killing while expression of 147-158 lacking the arginine at position 156(termed here as 147-155TG) does not, and that addition of a single amino acid, Met159, to the C terminus of the blocked peptide (creating 147-155TGM) restores presentation. (See, Yellen-Shaw, et. al., 1997 J Immunol. 158(4):1727-33).

Definitions

A “viral protein” includes any polypeptide encoded by.a viral gene. As used herein, “polypeptide” and “protein” are synonymous.

A “modified viral protein” includes a viral protein that has a different primary, secondary and tertiary amino acid sequence as compared to a unmodified viral protein (i.e., a wild-type viral protein). A modification to a viral protein or to a nucleic acid encoding a modified viral protein that disrupts the three dimensional structure of the protein, such that the proteolytic degradation of the modified viral protein is altered (e.g., increased or decreased.) Such modification includes an insertion, substitution or deletion of one or more amino acids, or an insertion, substitution or deletion of one or more nucleic acids in a nucleic acid sequence that encodes a viral protein, preferably in an internal, e.g., hydrophobic region of the protein.

A “modified nucleic acid” or “modified viral nucleic acid” includes a nucleic acid that encodes for a modified (viral) protein.

The “tertiary structure” of a polypeptide represents the three-dimensional structure of a polypeptide.

The “secondary structure” of a polypeptide represents the folding of the peptide chain into an alpha helix, beta pleated sheet, or random coil. The secondary structure of a polypeptide can be determined by applying one or more algorithms to the primary amino acid sequence of the polypeptides. These algorithms include the DPM method, the Homolog method, and the Predator method.

A “domain structure” of a viral protein includes any polypeptide derived from the viral protein that is at least one amino acid shorter in length than the viral protein. Generally, domain structures are structures that define the secondary structure of the polypeptide or affect the activity of the polypeptide binding to a ligand, recognition by an antibody, catalytic activity, or binding with other molecules.

An “internal region” of a polypeptide includes any amino acid of the polypeptide other than the N-terminal or C-terminal amino acid. An internal region of a polypeptide also includes one or more amino acids present in a hydrophobic domain of a polypeptide.

A “hydrophobic domain” of a polypeptide includes regions of the polypeptide that are inaccessible to solvent under physiological (e.g., non-denaturing) conditions.

“Antigen presentation” includes the expression of antigen on the surface of a cell in association with major histocompatability complex class I or class II molecules (MHC-I or MHC-II.) Antigen presentation is measured by methods known in the art. For example, antigen presentation is measure using an in vitro cellular assay as described in Gillis, et al., J. Immunol. 120: 2027 (1978).

“Immunogenicity” includes the ability of a substance to stimulate an immune response. Immunogenicity is measured, for example, by determining the presence of antibodies specific for the substance. The presence of antibodies is detected by methods known in the art, for example an ELISA assay.

“Proteolytic degradation” includes degradation of the polypeptide by hydrolysis of the peptide bonds. No particular length is implied by the term peptide. Proteolytic degradation is measured, for example, using electrophoresis (e.g., gel electrophoresis), NMR analysis or mass spectral analysis.

As used herein, a “virus” includes any infectious particle having a protein coat surrounding an RNA or DNA core of genetic material.

By a “portion” of the polypeptide is meant two or more amino acids of the polypeptide, and includes domains of the polypeptide (e.g., the intracellular, transmembrane or extracellular domains, signal peptides, and nuclear localization signals.) A portion includes any fragment of a polypeptide created by proteolytic cleavage.

“Antigen presenting cells” (APCs) capture and process antigens for presentation to T-lymphocytes, and produce signals required for the proliferation and differentiation of lymphocytes. APCs include somatic cells, B-cells, macrophages and dendritic cells (e.g., myeloid dendritic cells.)

The Immune Response to Influenza Virus

Both humoral and cellular immunity (mucosal and systemic) is involved in the control of influenza infection, with the humoral response playing a main role in natural infection. Local humoral response results in generation of neutralizing antibodies against HA and NA proteins secreted in the upper respiratory tract. Their production is imperative for the block of viral infection. Antibodies secreted locally in the upper respiratory tract are a major factor in resistance to natural infection. This includes both the production of secretory IgA and serum IgG. In addition, systemic cellular response produces cytotoxic T lymphocytes that eliminate virus-infected cells. Influenza viruses, as mentioned above, mutate and change antigenic sites of surface glycoproteins. Therefore, a previously infected organism's immune system will not recognize a novel viral strain and will not be protected against it. At the same time, the immune response induced by infection protects against reinfection with the same virus or an antigenically similar viral strain. Natural infection may lead to long-lasting immunity to the infecting virus, as demonstrated by the reappearance of the influenza A H1N1 subtype in 1977, when only subjects under the age of 20 years became infected.

The humoral immune system, including both the mucosal and systemic arms, plays a major role in immunity to natural influenza infection, while the cell-mediated response is particularly effective in clearing virus-infected cells. Immunity to influenza is a multifaceted phenomenon with virus virulence, innate immunity, specific IgG antibody, cell-mediated immunity and local antibodies being contributing factors. Generally, the primary cytotoxic response is detectable after 6-14 days and wanes by day 21 in infected or vaccinated individuals.

The present invention induces the cytotoxic T-cell response to generally be directed against conserved nucleoproteins NP and M1, and the non-structural protein NS-1 which, prior to the present invention, was not recognized as a vaccine candidate.

Nucleoprotein (NP)

Influenza A virus RNA segment 5 encodes NP (a polypeptide of 498 amino acids in length), which is rich in arginine, glycine and serine residues and has a net positive charge at neutral pH. The majority of the polypeptide has a preponderance of basic amino acids and an overall predicted pI of 9.3, but the C-terminal 30 residues of NP are, with a pI of 3.7, markedly acidic. In influenza B and C viruses, the length of the homologous NP polypeptide is 560 and 565 residues, respectively. Alignment of the predicted amino acid sequences of the NP genes of the three influenza virus types reveals significant similarity among the three proteins, with the type A and B NPs showing the highest degree of conservation. Phylogenetic analysis of virus strains isolated from different hosts reveals that the NP gene is relatively well conserved, with a maximum amino acid difference of less than 11% (See, Shu et al., J. Virology 67, 2723-2729). The nucleotide and amino acid sequence of the influenza A virus (A/Paris/908/97(H3N2)) nucleoprotein (NP) gene are provided in Table 1.

TABLE 1
Influenza NP nucleic acid and polypeptide sequences

atggcgtccc aaggcaccaa acggtcttat gaacagatgg aaactgatgg ggatcgccag
aatgcaactg agattagggc atccgtcggg aagatgattg atggaattgg gcgattctac
atccaaatgt gcactgaact taaactcagt gattatgaag ggcggttgat ccagaacagc
ttgacaatag agaaaatggt gctctctgct tttgatgaga gaaggaatag atatctggaa
gaacacccca gcgcggggaa agatcctaag aaaactggag ggcccatata caagagagta
gatggaagat ggatgaggga actcgtcctt tatgacaaag aagaaataag gcgaatctgg
cgacaagcca acaatggtga ggatgcgaca gctggtctaa ctcacatgat gatctggcat
tccaatttga atgatacaac ataccagagg acaagagctc ttgttcgcac cggaatggat
cccagaatgt gctctctgat gcagggctcg actctcccta gaaggtctgg agctgcaggt
gctgcagtca aaggaatcgg gacaatggtg atggagctga tcagaatggt caaacggggg
atcaacgatc gaaatttctg gagaggtgag aatgggcgga aaacaaggag tgcttatgag
agaatgtgca acattcttaa aggaaaattt caaacagctg cacaaagagc aatggtggat
caagtgagag aaagtcggaa cccaggaaat gctgagatcg aagatctcat atttttggca
agatctgcat taatattgag agggtcagtt gctcacaaat cttgcctacc tgcctgtgtg
tatggacctg cagtatccag tgggtacgac ttcgaaaaag agggatattc cttggtggga
atagaccctt tcaaactact tcaaaatagc caagtataca gcctaatcag accgaacgag
aatccagcac acaagagtca gctggtatgg atggcatgcc attctgctgc atttgaagat
ttaagattgt taagcttcat cagagggacc aaagtatctc cgcgggggaa actttcaact
agaggagtac aaattgcttc aaatgagaac atggataata tgggatcaag tactcttgaa
ctgagaagcg ggtactgggc cataaggacc aggagtggag gaaacactaa tcaacagagg
gcctccgcag gccaaatcag tgtgcaacct acgttttctg tacaaagaaa cctcccattt
gaaaagtcaa ccgtcatggc agcattcact ggaaatacgg agggaagaac ctcagacatg
agggcagaaa tcataagaat gatggaaggt gcaaaaccag aagaagtgtc tttccgtggg
cggggagttt tcgagctctc agacgagaag gcaacgaacc cgatcgtgcc ctcttttgac
atgagtaatg aaggatctta tttcttcgga gacaatgcag aagagtacga caattaa
(SEQ ID NO: 4, from GenBank Accession No. AF483604).
MASQGTKRSYEQMETDGERQNATEIRASVGKMIGGIGRFYIQMCTELKLSDYEGRLIQNSLTIER
MVLSAFDERRNKYLEEHPSAGKDPKKTGGPIYRRVNGKWMRELILYDKEEIRRIWEQANNGDDAT
AGLTHMNIWHSNLNDATYQRTRALVRTGMDPRNCSLMQGSTLPRRSGAAGAAVKGVGTMVNELVR
NIKRGINDRNFWRGENGRKTRIAYERNCNILKGKFQTAAQKAMMDQVRESRNPGNAEFEDLTFLA
RSALILRGSVAHKSCLPACVYGPAVASGYDFEREGYSLVGIDPFRLLQNSQVYSLIRPNENPAHK
SQLVWMACHSAAFEDLRVLSFIKGTKVLPRGKLSTRGVQIASNENMETMESSTLELRSRYWAIRT
RSGGNTNQQRASAGQISIQPTFSVQRNLPFDRTTIMAAFNGNTEGRTSDMRTEIIRMMESARPED
VSFQGRGVFELSDEKAASPIVPSFDMSNEGSYFFGDNAEEYDN
(SEQ ID NO: 1)

M1

RNA segment 7 of the influenza virus A genome encodes for two proteins: M1 (matrix 1) and M2 (matrix 2). The M1 is a relatively small, highly conserved protein (252 amino acids [aa] in type A and 248 aa in type B viruses). M1 is the most abundant protein in virus particle and plays critical roles in many aspects of virus replication. These include (i) dissociation of M1 from the M1/viral ribonucleoprotein (vRNP) complex during the entry and uncoating of infecting virus, (ii) nuclear entry of M1, (iii) interaction of M1 with vRNP to form M1/vRNP complex, (iv) role of M1 in the exit of vRNP from the nucleus into the cytoplasm, (v) interaction of M1 with viral envelope proteins (hemagglutinin [HA], neuraminidase, and ion channel M2), (vi) membrane binding of M1, (vii) dimer/oligomer formation of M1, (viii) role of M1 in virus budding, including recruitment of viral components at the assembly site and recruitment of host components for budding and release of virus particles. M1 protein is encoded by an mRNA that is colinear, while M2 protein is synthesized from spliced mRNA. M1 protein possesses multiple functional motifs, such as in the helix 6 (H6) domain (amino acids 91 to 105), including a nuclear localization signal (NLS) (101-RKLKR-105) that is involved in translocating M1 from the cytoplasm into the nucleus. M2 protein is a transmembrane protein composed of three domains: 1) 24 residues representing the N-terminal region, 2) 19 hydro-phobic residues that serve as a membrane anchor, and 3) 54 residues near the C-terminal in the cytoplasmic domain. The M2 protein has been found to play a role in influenza replication and assembly of virion particles. Further experimentation has demonstrated that this protein is an acid-activated ion channel for virus replication.

Influenza M1 nucleic acid and polypeptide sequences are shown in Table 2.

TABLE 2

Influenza M1 nucleic acid and
polypeptide sequences

atgagtcttctaaccgaggtcgaaacgtacgttctctctatcgtcccgtc
aggccccctcaaagccgagatcgcgcagagacttgaagatgtctttgctg
ggaagaacaccgatctcgaggcactcatggaatggctaaagacaagacca
atcctgtcacctctgactaaggggattttaggatttgtgttcacgctcac
cgtgcccagtgagcgaggactgcagcgtagacgctttgtccagaatgccc
ttaatgggaatggggatccaaacaacatggacagggcagtgaaactgtac
aggaagctcaaaagggaaattacattccacggggccaaagaagtagcgct
cagttattctactggtgcacttgccagctgcatgggcctcatatacaaca
gaatggggactgtaaccactgaagtggcatttggcctagtgtgtgccact
tgtgagcagattgccgactcccagcatcggtcccacagacagatggtgac
gacaaccaacccactaatcagacatgagaacaggatggtgctggccagta
ccacggctaaggccatggagcagatggcagggtcgagtgaacaggcagca
gaagccatggaggttgctagtcaggctaggcagatggtgcaggcaatgag
gaaccattgggactcaccctagctccagtgccggtctaaaagatgatctt
cttgaaaatttgcaggcctaccagaaacggatgggagtgcaaatgcagcg
attcaagtgatcctctcgttattgccgcaagcatcattgggatcttgcac
ttgatattgtggattcttgatcgtcttttcttcaaatgcatttatcgtcg
ccttaaatacggtttgaaaagagggccttctacggaaggagtgcctgagt
ctatgagggaagagtatcggcaggaacagcagagtgctgtggatgttgac
gatagtcattttgtcaacatagagctggagtaaaaaa
(Influenza A M1 and M2 encoding genes;
SEQ ID NO: 5, from GemBank Accession
No. AY303656)
MSLLTEVETYVLSIVPSGPLKAEIAQRLEDVFAGKNTDLEALMEWLKTRP
ILSPLTKGILGFVFTLTVPSERGLQRRRFVQNALNGNGDPNNNDRAVKLY
RKLKREITFHGAKEVALSYSTGALASCMGLIYNRMGTVTTEVAFGLVCAT
CEQIADSQHRSHRQMVTTTNPLIRHENRNVLASTTAKAMEQMAGSSEQAA
EANEVASQARQMVQANRTIGTHPSSSAGLKDDLLENLQAYQKRNGVQMQR
FK
(M1-A polypeptide from GenBank Accession No.
AY303656; SEQ ID NO: 2)
MSLFGDTIAYLLSLTEDGEGKAELAEKLHCWFGGKEFDLDSALEWIKNKR
CLTDIQKALIGASICFLKPKDQERKRRFITEPLSGMGTTATKKKGLILAE
RKMRRCVSFHEAFEIAEGHESSALLYCLMVMYLNPGNYSMQVKLGTLCAL
CEKQASHSHRAHSRAARSSVPGVRREMQMVSAMNTAKTMNGMGKGEDVQK
LAEELQSNIGVLRSLGASQKNGEGIAKDVMEVLKQSSMGNSALVKKYL
(M1-B polypeptide from GemBank Accession No.
AB036877; SEQ ID NO: 7)

Nonstructural Protein 1 (NS1)

Nonstructural protein 1 (NS 1) of influenza A virus is the nonstructural protein encoded by the shortest of the eight RNA segments comprising the fragmented RNS genome of this Othomyxoviridae representative. NS1 consists of approximately 230 amino acids (e.g., 237 amino acids) and has been suggested and at least partially proven to perform several important functions that enable effective replication of the virus in its host. First, NS1 has been shown to inhibit the host mRNA's processing mechanisms, specifically host mRNA adenylation, by binding to the poly(A) tail of mRNA, preventing nuclear export and pre-mRNA splicing (via its C-terminus). Second, it increases the level of translation of viral RNAs (via its central domain). Thus, NS1 protein can antagonize the production of cellular proteins at several levels—transcriptional, post-transcriptional and translational.

Moreover, NS1 is capable of binding dsRNA (this function has been mapped to the N-terminal 73 amino acids, strict RNA-binding domain is delineated as spanning amino acids 19-38) (41) and interacting with a putative cellular kinase and thus preventing the activation of the interferon (IFN)-inducible dsRNA-activated kinase, 2′,5′-oligoadenylate synthetase system, and cytokine transcription factors (NF-κB, IRF-3 and c-Jun/ATF-2). Consequently, NS1 protein inhibits the expression of IFN-α and -β, delays the development of apoptosis in infected cells, and prevents the formation of the antiviral state in neighboring cells. Needless to say, that IFN-α and -β serve as the first line of antiviral defense (innate immune response), being synthesized within hours of infection.

NS1 is essential for influenza virus A replication and the corresponding deletion or truncation mutants of NS1 can replicate only in those cellular systems that lack IFN induction systems, such as the Vero cell line, 6-day-old eggs, STAT1 [symbol] mice or PKR [symbol] mice. NS1 truncation mutant encoding the first 125 amino acids of protein, thus lacking the C-terminal domain, has also been shown to be as effective as the wild-type virus in the suppression of IL-1β and IL-18 production in virus-infected macrophages, but at the same time was not able to inhibit the production of numerous antiviral cytokines, such as IFN-β, IL-6, TNF-α and MIP-1α. In the same study, another group of NS1 mutants possessing impaired RNA-binding and dimerization domains induced higher levels of biologically active IL-1βand IL-18. Thus, in a primary human macrophage system, NS1 functions as a main modulator of the production of pro-inflammatory cytokines. Generally, it is accepted that the adverse interaction of the NS1 protein with the antiviral immune defense of the cell plays a major role in augmentation of influenza virulence and as a consequence, NS1 also likely functions as a main regulator of virus replication in the host.

Recent data point to the possibility that the virulence of especially pathogenic influenza viruses, including H5N1 avian influenza virus (which by the middle of 2004 had killed 6 out of 18 people infected; preliminary data compiled as of May 1, 2005 put this numbers as 54 percent out of 109 people infected) and human H1N1 virus that is thought to be the infectious agent of the 1918 pandemic (which caused an estimated 20-40 million deaths worldwide) bore specific changes in NS1, which may at least be partially responsible for their markedly increased pathogenicity. Studies using artificially created reassortants containing the NS1 gene from highly pathogenic Hong Kong H5N1/97 and avian 2001 H5N1 strains, indicate that NS1 is, at least in part, responsible for the imbalance of inflammatory cytokines observed in vivo.

NS1 protein does not constitute a part of the virion, but is produced early (well before the expression of M1 and HA) and abundantly during the infection process and is accumulated in the nucleus and later in the cytoplasm of infected cells. A humoral immune response to NS1 has been observed in the sera of animals experimentally infected with live virus, but not in the sera of those immunized with inactivated or live-attenuated virus strains (since in most of the attenuated strains it is indeed NS1 that is incapacitated). CTL responses against NS1 were detected in PBMC from healthy donors from the general population. This testifies to the generation of an anti-NS1 cellular response throughout the normal course of disease and to the existence of strong immunologic memory against this protein. Furthermore, a single particular change (amino acid 127) in the NS1 CTL epitope has been linked to a higher level of viral expression. This may point to the existence of CTL-directed evolutionary pressure against this protein and thus indirectly suggest that the strong immune response against this protein may hinder viral infection. The early studies of immune response against various influenza vaccine preparations containing partial or full-length NS1 product also testified to the beneficial activity induced by this protein in experimentally infected animals.

Influenza NS-1 nucleic acid and polypeptide sequences are shown in Table 3. Additional NS-1 polypeptide sequences are available at, e.g., GenBank accession numbers NP_{—}056666, AAA43756, AAA43688, AAA43139, AAA43132, AAA43124, AAA43121, and AAA43086.

TABLE 3
Influenza NS-1 polypeptide sequence

MDSNTVSSFQVDCFLWHVRKRFADQELGDAPFLDRLRRDQKSLRGRGSTL
GLDIETATRAGKQIVERILEEESDEALKMTIASVPASRYLTDMTLEEMSR
DWFMLMPKQKVAGSLCIRMDQAIMDKNIILKANFSVIFDRLETLILLRAF
TEEGAIVGEISPLPSLPGHTDEDVKNAIGVLIGGLEWNDNTVRVSETLQR
FAWRSSNEDGRPPLPPKQKRKMARTIESEV
(NS-1 polypeptide from GenBank Accession No.
AAA43130; SEQ ID NO: 3, Influenza A virus (A/Anas
acuta/Primorje/695/76(H2N3))

Modified Viral Polypeptides

The present invention relates, in part, to modified viral polypeptides (and nucleic acids encoding them for expression in cells) that contain a modification in the polypeptide sequence. The disruptive element results in a conformational change in the modified polypeptide structure, such that the proteolytic processing of the modified polypeptide is different from that of the unmodified polypeptide. Without wishing to be bound by theory, one mechanism of action for the difference in proteolytic processing is that the conformational change alters (e.g., increases or decreases) the accessibility of internal amino acids. Proteolytic processing occurs via the proteasome. Alternatively, proteolytic processing occurs via non-proteasomal pathways.

In embodiments of the invention, one or more hydrophobic amino acids of an influenza NP, M1 or NS-1 protein are replaced by one or more hydrophilic amino acids. Alternatively, one or more hydrophilic amino acids are inserted into the core domain of the influenza protein. Table 4 lists representative hydrophobic and hydrophilic amino acids (i.e., those amino acids that are not hydrophobic, including positively and negatively charged amino acids).

TABLE 4
Amino acid characteristics

| Amino acid | Hydrophobic | Positive | Negative | Polar | Charged | Codon |
|---|---|---|---|---|---|---|
| Alanine | X | — | — | — | — | GCU, GCC, GCA, |
| | | | | | | GCG |
| Cysteine | X | — | — | — | — | UGU, UGC |
| Aspartate | — | — | X | X | X | GAU, GAC |
| Glutamate | — | — | X | X | X | GAA, GAG |
| Phenylalanine | X | — | — | — | — | UUU, UUC |
| Glycine | X | — | — | — | — | GGU, GGC, GGA, |
| | | | | | | GGG |
| Histidine | X | X | — | X | X | CAU, CAC |
| Lysine | — | X | — | X | X | AAA, AAG |
| Isoleucine | X | — | — | — | — | AUU, AUC, AUA |
| Leucine | X | — | — | — | — | UUA, UUG, CUU, |
| | | | | | | CUC, CUA, CUG |
| Methionine | X | — | — | — | — | AUG |
| Asparagine | — | — | — | X | — | AAU, AAC |
| Proline | — | — | — | — | — | CCU, CCC, CCA, CCG |
| Glutamine | — | — | — | X | — | GGU, GGC, GGA, |
| | | | | | | GGG |
| Arginine | — | X | — | X | X | CGU, CGC, CGA, |
| | | | | | | CGG, AGA, AGG |
| Serine | — | — | — | X | — | UCU, UCC, UCA, |
| | | | | | | UCG, AGU, AGC |
| Threonine | X | — | — | X | — | ACU, ACC, ACA, |
| | | | | | | ACG |
| Valine | X | — | — | — | — | GUU, GUC, GUA, |
| | | | | | | GUG |
| Tryptophan | X | — | — | X | — | UGG |
| Tyrosine | X | — | — | X | — | UAU, UAC |

Preferred modified viral polypeptides include modified influenza NP polypeptides, non-limiting examples of which are provided in Table 5.

TABLE 5
Modified NP polypeptides

| | Corresponding | | |
|---|---|---|---|
| | amino acids of | | |
| Target NP peptide | SEQ ID NO: 2 | Amino acid substitutions¹ | Amino acid Insertions² |
| FYIQMCT | 39-45 | ³⁹FYDQMCT⁴⁵ | ³⁹FDDYIQMCT⁴⁵ |
| | | ³⁹FYIQDDT⁴⁵ | |
| SLTI | 60-63 | ⁶⁰SUTI⁶³ | ⁶⁰SDDLTI⁶³ |
| RRIWR | 117-121 | ¹¹⁷RRDDR¹²¹ | ¹¹⁷RDDRIWR¹²¹ |
| TMVMELVRMIKR | 188-199 | ¹⁸⁸TMVMEDDRMIKR¹⁹⁹ | ¹⁸⁸TMVMEDDLVRMIKR¹⁹⁹ |
| | | ¹⁸⁸TMVMELVRDDKR¹⁹⁹ | |
| NAEFEDLTFLARSALILRGSV | 250-270 | ²⁵⁰NAEFEDLTDDARSALILRGSV²⁷⁰ | ²⁵⁰NAEFEDLTFDDLARSALILRGSV²⁷⁰ |
| | | ²⁵⁰NAEFEDLTFLARSADDDRGSV²⁷⁰ | |
| QLVWMACHSAAFE | 327-339 | ³²⁷QLVDDACHSAAFE³³⁹ | ³²⁷QLVDDWMACHSAAFE³³⁹ |
| | | ³²⁷QLVWDDCHSAAFE³³⁹ | ³²⁷QLVWMACHSAADDFE³³⁹ |
| | | ³²⁷QLVWMDDHSAAFE³³⁹ | |
| | | ³²⁷QLVWMACHSADDE³³⁹ | |
| MRTEIIRMMES | 440-450 | ⁴⁴⁰MRTEDDRMMES⁴⁵⁰ | ⁴⁴⁰MRTEDDIIRMMES⁴⁵⁰ |
| | | ⁴⁴⁰MRTEIIRDDES⁴⁵⁰ | |

¹Substituted amino acids are in bold and underlined.
²Inserted amino acids are in bold and underlined.

Additional modified viral polypeptides include modified influenza M1 polypeptides, non-limiting examples of which are provided in Table 6.

TABLE 6
Modified M1 polypeptides

| | | |
|---|---|---|
| | ▾ ▾ ▾ | |
| M1-A | MSLLTEVETYVLSIIPSGPLKAEIAQRLEDVFAGKNTDLEVLMEWLKTRPILSPLTKGIL | (60) |
| M1-B | MSLFGDTIAYLLSLIEDGEGKAELAEKLHCWFGGKEFDLDSALEWIKNKRCLTDIQKALI | (60) |
| | ***: :. :*:**:* .* ***:*::*. *.**: **: :**:*.: *: : *.:: | |
| M1-A | GFVFTLTVPSERGLQRRRFVQNALNGNGDPNNMDKAVKLYRKLKRE-ITFHGAKEISLSY | (119) |
| M1-B | GASICFLKPKDQ-ERKRRFITEPLSGMGTTATKKKGLILAERKMRRCVSFHEAFEIAEGH | (119) |
| | * : : *.:: ::***: :.*.* * . . .*.: * .: * ::** * **: .: | |
| | ↓ (158) end of X-Ray structure | |
| M1-A | SAGALASCMGLIYNRMGAVTTEVAFGLVCATCEQIADSQHRSHRQMVTTTNPLIRHENRM | (179) |
| M1-B | ESSALLYCLMVMYLNPENYSMQVKLGTLCALCEKQASHSHRAHSRAARSSVPGVRREMQM | (179) |
| | .:.** *: ::* . : :* :* :** **: *. .**:* : . :: * :*:* :* | |
| M1-A | VLASTTAKANEQMAGSSEQAAEAMEVASQARQMVQAMRTIGTHPSSSAGLKNDLLENLQA | (239) |
| M1-B | VSAMNTAKTMNGMG----KGEDVQKLAEELQNNIGVLRSLGASQKNGEGIAKDVMEVLKQ | (235) |
| | * * .***:*: *. :. :. ::*.: :: : .:*::*: ... *: :*::* *: | |
| M1-A | YQKRMGVQMQRFK (252) (SEQ ID NO: 10) | |
| M1-B | SSMGNSALVRKYL (248) (SEQ ID NO: 11) | |
| | . .. :::: | |

¹Amino acid insertion sites are indicated by downward pointing arrowheads.

The modification to the influenza protein may include a disruptive element, as described in pending U.S. patent applications Ser. No. 10/866,484, filed Dec. 19, 2003 and U.S. patent applications Ser. No. 10/741,466, filed Jun. 11, 2004, the contents of which are incorporated herein in their entireties.

Influenza Polypeptide Consensus Sequences

As described above, the NP and M1 polypeptides are generally conserved among strains of influenza A virus. Multiple sequence alignment, such as performed using ClustalW analysis, provides consensus polypeptide sequences for NP, M1 and NS-1 as described in the following tables.

TABLE 7
Multiple sequence alignment of influenza A nucleoproteins

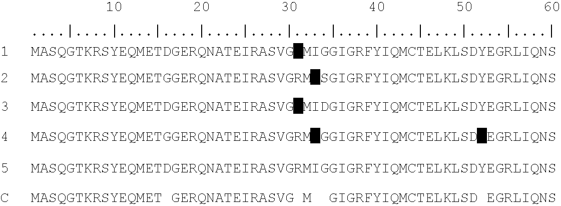

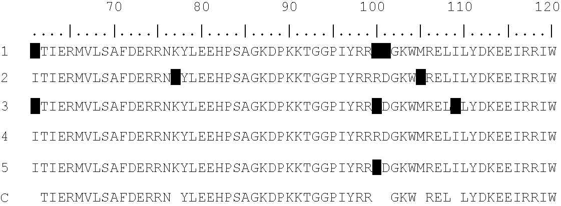

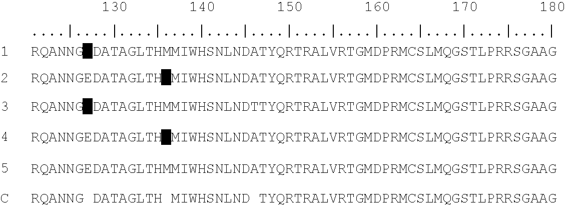

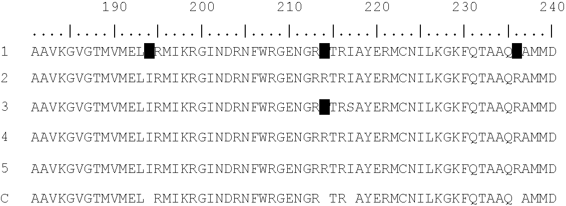

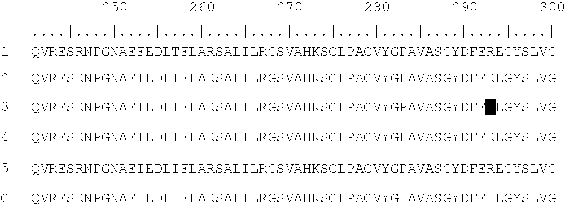

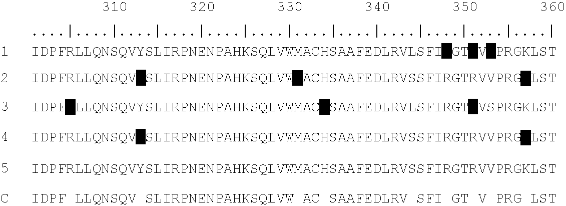

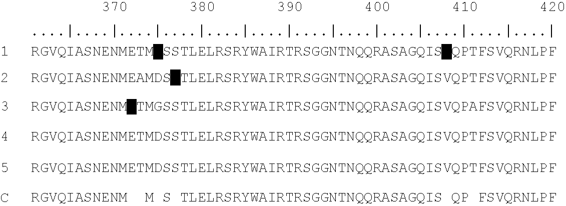

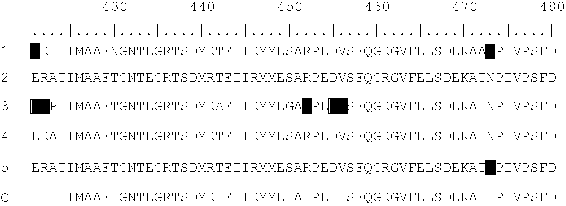

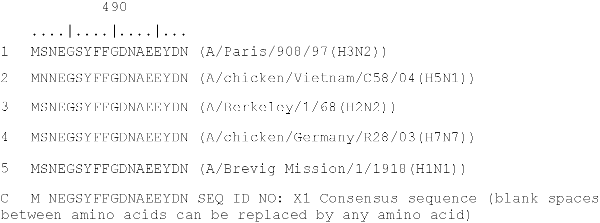

TABLE 8
Multiple sequence alignment of influenza A M1 proteins

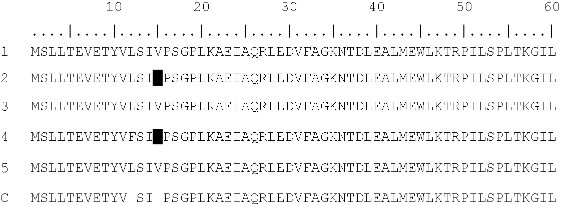

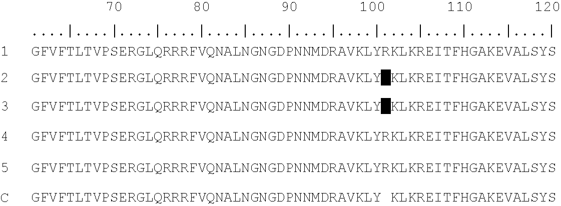

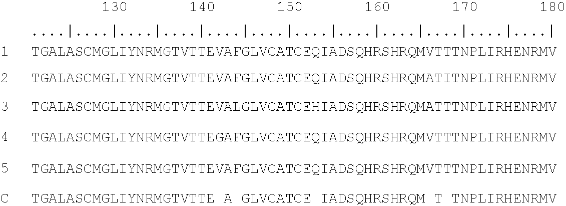

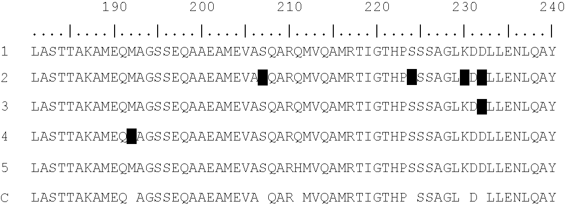

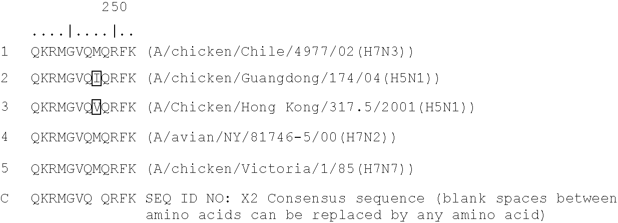

TABLE 9
Multiple sequence alignment of influenza A NS-1 proteins

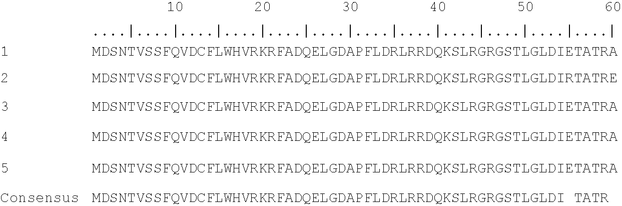

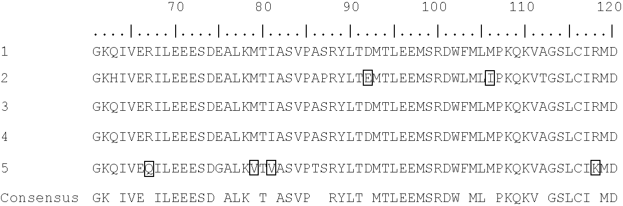

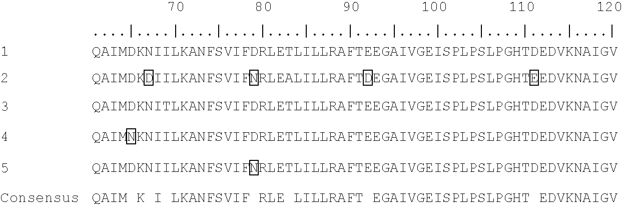

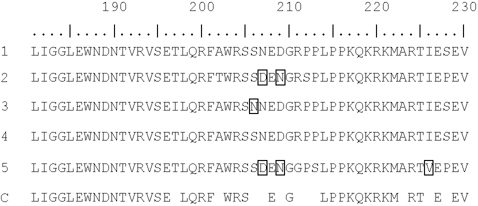

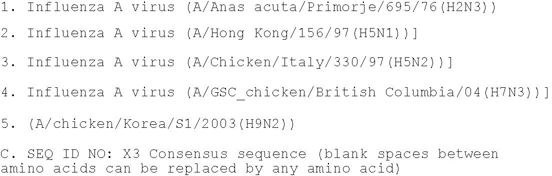

Influenza Immunogenic Peptides Derived from Influenza NP, M1 or NS-1 Polypeptides

The invention also provides vaccines that contain immunogenic peptides derived from an influenza protein, such as NP, M1 and NS-1. Exemplary immunogenic peptides are provided in Table 10. Also see, Boon et al. J Virol. 2002. Vol. 76(2):582-90; Terajima et al. Virology. 1999. Vol. 259(1):135-40; Jameson et al. J Immunol. 1999. Vol. 162(12):7578-83; and Jameson et al. J Virol. 1998. Vol. 72(11):8682-9.

TABLE 10
influenza immunogenic peptides

| Influenza protein | peptide sequence | location of peptide |
|---|---|---|
| NS1 | DRLRRDQKS | 34-42 |
| | AIMDKNIIL | 122-130 |
| NP epitopes | CTELKLSDY | 44-52 |
| | RRSGAAGAAVK | 174-184 |
| | EDLTFLARSAL | 254-264 (255-265) |
| | ILRGSVAHK | 265-273 |
| | ELRSRYWAI | 380-388 |
| | SRYWAIRTR | 383-391 |
| M1 epitopes | SGPLKAEIAQRLEDV | 17-31 |
| | GILGFVFTL | 58-66 |
| | ASCMGLIY | 128-135 (125-132) |

Expression Vectors Encoding Modified Polypeptides

The nucleic acid encoding the modified polypeptide is in a suitable expression vector. By suitable expression vector is meant a vector that is capable of carrying and expressing a complete nucleic acid sequence coding for the modified polypeptide. Such vectors include any vectors into which a nucleic acid sequence as described above can be inserted, along with any preferred or required operational elements, and which vector can then be subsequently introduced or transferred into a host organism and replicated in such organism. The vector can be introduced by way of transfection or infection. Preferred vectors are those whose restriction sites have been well documented and which contain the operational elements preferred or required for transcription of the nucleic acid sequence. The vectors include retroviral vectors, adenoviral vectors, lentiviral vectors, plasmid vectors, cosmid vectors, bacterial artificial chromosome (BAC) vectors, and yeast artificial chromosome (YAC) vectors.

To construct the vector of the present invention, it should additionally be noted that multiple copies of the nucleic acid sequence encoding modified polypeptide and its attendant operational elements may be inserted into each vector. In such an embodiment, the host organism would produce greater amounts per vector of the desired modified polypeptide. In a similar fashion, multiple different modified polypeptides may be expressed from a single vector by inserting into the vector a copy (or copies) of nucleic acid sequence encoding each modified polypeptide and its attendant operational elements.

Preferred vectors are those that function in a eukaryotic cell. Examples of such vectors include, but are not limited to, plasmids, viral vectors including vaccinia virus, adenovirus, adeno-associated, VEEV, Sendai-based, NDV-based or DNA constructs practiced in the art. Preferred vectors include vaccinia viruses. While the present invention provides three vectors, it should be understood that one or more vectors can be used for the vaccine of the invention.

Confirmation of the modification of three-dimensional structure of the polypeptide is determined by methods known in the art. For example, computer aided molecular modeling (e.g., spherical harmonics), or crystallographic analysis may be used. Alternatively, NMR or mass spectral analyses of modified polypeptides or peptide fragments thereof are performed. Further, the modified polypeptide is contacted with one or more proteolytic enzymes (e.g., proteasomal) that have differential activity (i.e., the proteolytic enzymes have a greater or reduced proteolytic activity) on the modified polypeptide in relation to the unmodified polypeptide.

The present invention provides a method of immunization comprising administering an amount of the modified polypeptide or a nucleic acid encoding the modified polypeptide (i.e., vaccine) effective to elicit a T cell response. Such T cell response can be measured by a variety of assays including ⁵¹Cr release assays (Restifo, N. P. J of Exp. Med., 177: 265-272 (1993)). The T cells capable of producing such a cytotoxic response may be CD8⁺ T cells, CD4⁺ T cells, or a population containing CD8⁺ T cells and CD4⁺ T cells.

Administration of Nucleic Acids Encoding Modified Polypeptides

The vaccine may be administered in combination with other therapeutic ingredients including, e.g., γ-interferon, cytokines, chemotherapeutic agents, or anti-inflammatory or anti-viral agents.

The vaccine can be administered in a pure or substantially pure form, but it is preferable to present it as a pharmaceutical composition, formulation or preparation. Such formulation comprises a modified polypeptide or a nucleic acid encoding the modified polypeptides together with one or more pharmaceutically acceptable carriers and optionally other therapeutic ingredients. Other therapeutic ingredients include compounds that enhance antigen presentation, e.g., gamma interferon, cytokines, chemotherapeutic agents, or anti-inflammatory agents. The formulations may conveniently be presented in unit dosage form and may be prepared by methods well known in the pharmaceutical art.

Formulations suitable for intravenous, intramuscular, intranasal, oral, subcutaneous, or intraperitoneal administration conveniently comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g., 0.1-2.0M), glycine, and the like, and having a buffered pH compatible with physiological conditions to produce an aqueous solution, and rendering the solution sterile. These may be present in unit or multi-dose containers, for example, sealed ampoules or vials.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057).

The formulations of the present invention may incorporate a stabilizer. Illustrative stabilizers are polyethylene glycol, proteins, saccharide, amino acids, inorganic acids, and organic acids which may be used either on their own or as admixtures. Two or more stabilizers may be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of 5.0-9.0, preferably within the range of 6-8.

When oral preparations are desired, the compositions may be combined with typical carriers, such as lactose, sucrose, starch, talc magnesium stearate, crystalline cellulose, methyl cellulose, carboxymethyl cellulose, glycerin, sodium alginate or gum arabic among others.

The method of immunization may comprise administering a nucleic acid sequence capable of directing host organism production of the modified polypeptide in an amount effective to elicit a T cell response. Such nucleic acid sequence may be inserted into a suitable expression vector by methods known to those skilled in the art. Expression vectors suitable for producing high efficiency gene transfer in vivo include retroviral, adenoviral and vaccinia viral vectors. The operational elements of such expression vectors are known to one skilled in the art. A preferred vector is vaccinia virus.

Expression vectors containing a nucleic acid sequence encoding modified polypeptide can be administered intravenously, intranasally, intramuscularly, subcutaneously, intraperitoneally or orally. A preferred route of administration is oral, intranasal or intramuscular.

The modified polypeptides and expression vectors containing nucleic acid sequence capable of directing host organism synthesis of modified polypeptides may be supplied in the form of a kit, alone, or in the form of a pharmaceutical composition.

Expression vectors include one or more regulatory sequences, including promoters, enhancers and other expression control elements (e.g., polyadenylation) signals. Such regulatory sequences are described, for example, in Goeddel, GENE EXPRESSION TECHNOLOGY: METHODS IN ENZYMOLOGY 185, Academic Press, San Diego, Calif. (1990).

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

The invention also provides a vaccine for immunizing a mammal against cancer, viral infection, bacterial infection, parasitic infection, or autoimmune disease, comprising a modified polypeptide or an expression vector containing nucleic acid sequence capable of directing host organism synthesis of modified polypeptide in a pharmaceutically acceptable carrier. In an alternative embodiment, multiple expression vectors, each containing nucleic acid sequence capable of directing host organism synthesis of different modified polypeptides, may be administered as a polyvalent vaccine.

Vaccination can be conducted by conventional methods. For example, a modified polypeptide can be used in a suitable diluent such as saline or water, or complete or incomplete adjuvants. The vaccine can be administered by any route appropriate for eliciting T cell response, such as intravenous, intraperitoneal, intramuscular, and subcutaneous. The vaccine may be administered once or at periodic intervals until a T cell response is elicited. T cell response may be detected by a variety of methods known to those skilled in the art, including but not limited to, cytotoxicity assay, proliferation assay and cytokine release assays.

The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient.

The present invention also includes a method for treating viral infection by administering pharmaceutical compositions comprising a modified polypeptide or an expression vector containing nucleic acid sequence capable of directing host organism synthesis of a modified polypeptide in a therapeutically effective amount. Again as with vaccines, multiple expression vectors may also be administered simultaneously. When provided therapeutically, the modified polypeptide or modified polypeptide-encoding expression vector is provided at (or after) the onset of the infection or at the onset of any symptom of infection or disease caused by virus. The therapeutic administration of the modified polypeptide or modified polypeptide-encoding expression vector serves to attenuate the infection or disease.

A preferred embodiment is a method of treatment comprising administering a vaccinia virus containing nucleic acid sequence encoding modified polypeptide to a mammal in therapeutically effective amount.

### EXAMPLES

### Example 1

### DNA Vaccination of Mice With a Vaccine Containing Influenza NP, M1 and NS1 Expressing Plasmids.

Generation of NP, M1 and NS1 Expression Plasmids.

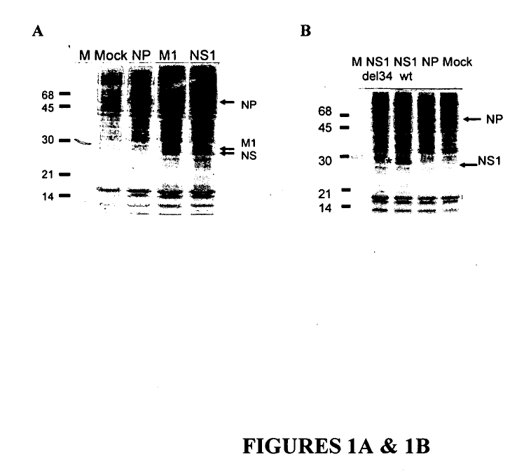

Expression plasmids carrying conserved influenza NP, M1 or NS1 genes were constructed by insertion of the PCR-amplified full viral gene sequences into the EcoRI site of pCAGGS vector (See, Niwa et al, (1991) Gene. 108:193-199.). The following viral sequences were used: NP from influenza strain A/WSN/33-H1N1, M1 from influenza strain A/WSN/33-H1N1, and NS1 from influenza strain A/PR/8/34-H1N1. The highly efficient pCAGGS vector possesses a composite promoter derived from CMV and the chicken actin gene, which was used for viral gene expression. The resulting constructs efficiently expressed NP, M1 and NS1 proteins in the human mammalian cell line 293T (FIG. 1A). Semi-confluent cultures of 293T cells were transfected with plasmid DNAs using Lipofectamine 2000 (Invitrogen) according to the manufacturer's directions. Either 1 or 2.7 μg of DNA and 7 μg of Lipofectamine2000 were used per one 3 cm diameter cell culture dish. All three influenza virus proteins could be visualized as the major bands upon subsequent SDS-PAGE analysis of 293T total cell extracts transiently transfected with the vectors described above (50 hours after transfection).

In order to create a recombinant inactivated NS1-expressing construct, two deletion mutants of NS1 in the same pCAGGS vector were generated by two sequential PCR reactions employed for the specific site-directed mutagenesis of NS1. Briefly, NS1-plasmid was used as a template for PCR with NS1-specific primers, Pfu or Turbo DNA polymerase and Dpn-I treatment procedure as per the manufacturer's directions (Stratagene). The first mutant was designed to contain the deletion of amino acids 34-41 (designated below as del34/41) and the second one to contain a double deletion of amino acids 34-41 and 184-188 (designated as del34/184).

To create the mutants as described above we have used the following groups of primers. For NS1 del34/41 mutant: 5′-GGT GAT GCC CCA TTC CTT TCC CTA AGA GGA AGG GGC AGC-3′ (forward); 5′-GCC CCT TCC TCT TAG GGA AAG GAA TGG GGC ATC ACC TAG-3′ (reverse). For the introduction of 184-188 deletion: 5′-GCA GTT GGA GTC CTC ATC GGA GAT AAC ACA GTT CGA GTC TC-3′ (forward) and 5′-GAG AC TCG AAC TGT GTT ATC TCC GAT GAG GAC TCC AAC TGC-3′. For selection and verification of positive mutants clones we used two additional deletion-specific primers: NS1/150 5′-AT CGG CTT CGC CGA GAT CAG-3′ (forward) and NS1/578 5′-GTT ATC ATT CCA TTC AAG TC-3′ (reverse). These two deletion-specific primers were used with two additional NS1-specific primers: NS1/719 (5′-CTG ATG AAT TCA AAC TTC TGA CCT-3′, reverse) and NS1/atg/(5′-ACC AAC TCG AGA TGG ATC CAA ACA CTG-3′, forward), respectively, using as a template samples of different plasmid DNAs isolated from site-mutated clones.

Subsequently, these novel recombinant NS1-containing constructs (pNS1del34 and pNS1del34/184) were tested for their expression in 293T cells. Plasmid pNS1 wt was used as a control. Experimental analysis demonstrated that the expression of at least one of these novel mutant NS1 proteins, NS1del34, is severely impaired (FIG. 1B). Therefore, in further experiments the immunological detection of mutant NS1 forms was employed.

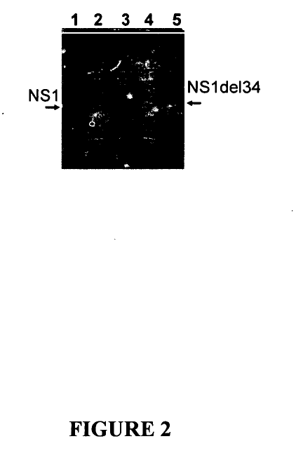

Polyclonal serum generated by guinea pig immunization with the whole NS1 molecule was used for protein detection by Western blotting. These results are shown in FIG. 2. The expression of wild- type NS1 protein (MW=27 K) was clearly seen in 293T cells transfected with pNS1 wt. Only a minor band of the NS1 del34-41 protein form was detected in cells transfected with pNS1del34, confirming the results presented in FIG. 1B. However, no NS1-specific bands were revealed in 293T cells transfected with the double mutant NS1del34/184.

Quantitative comparison of both bands showed that the expression of mutant NS1/del34-41 in this cell system was more than 25 times lower than that of NS1 wt. When the proteosomal stability of NS1 wt and NS1del34-41 was assayed using proteosomal inhibitor MG132, there was no additional stabilization and protein accumulation seen for both of the NS1 forms. This indicates that the underlying reason for the low levels of NS1 del34-41 protein is a function of its expression mechanisms and not due to the instability of the designed mutant.

Immunization with NP, M1 and NS1 combination in vivo.

X11-Blue E. Coli cells were transformed with the four plasmids described above, grown overnight and plasmid DNAs were subsequently purified with EndoTox-free Kit (V-gene; Canada). Concentration of plasmid DNA stocks and DNA quantities for the animal injections were calculated based on OD at 260 nm. 4 μg of each plasmid was injected intramuscularly per mouse per vaccination. The experimental setup for the vaccination studies in vivo was as following. Animals were divided into three groups (29 mice in each): control group (or group 1, injected with empty pCAGGS vector DNA), group 2 (injected with a mixture of three plasmids expressing wild-type conserved influenza proteins pNP wt, pM1 wt and pNS1 wt) and group 3 (injected with plasmids pNP wt, pM1 wt and pNS1del34). Mice were subjected to the immunization with plasmid DNAs three times with 14 days intervals in between. Two immune response characteristics were monitored: anti-viral CTL response and antibody generation.

CTL response in vivo. Mice of 10-12 g weight were injected intramuscularly with plasmid DNA three times at 14 days interval. Mice in the placebo group were inoculated with pCAGGS vector DNA. Six days after the third vaccination, three mice from each group were sacrificed, and their spleen cells were purified by the ficoll-verografin centrifugation procedure. Approximately, 10₈ isolated cells were stimulated in vitro by co-cultivation at 10:1 ratio with the syngeneic spleen cells infected with influenza A/PR/8/34 (H1N1) virus. These feeder cells were prepared from healthy mice and infected in vitro with influenza A/PR/8/34 at MOI 20 PFU per cell for 24 hours and inactivated by UV irradiation for 10 min. High levels of NP, M1 and NS1 expression in target spleen cells was demonstrated by immunoblotting with virus protein specific antibodies.

Splenocytes, isolated from mice infected intranasally twice at three-week intervals with a sublethal dose of influenza A/Aichi/2/68 (H3N2) virus, were used as a positive CTL control. Stimulated splenocytes were incubated in DMEM containing FCS (10%) and 2-mercaptoethanol (2 μM) for 16 days. Mouse mastocytoma cells p815 infected with influenza A/PR/8/34 virus (MOI 20 PFU per cell) for 24 hrs were used as a target, and cytotoxic activity was measured by lactate dehydrohenase (LDH) release (CytoTox 96 Kit; Promega). Target p815 infected cells (0.3×10⁵) were mixed with two-fold dilutions of stimulated effector cells starting with 3.0×10⁶ cells and incubated in 100 μl volume for 6 hrs at 37° C. CTL activity (as % of cell lysis) was calculated by the following formula:
(experimental release-spontaneous release)/(maximum release-spontaneous release)×100. Target cells incubated in medium only or with medium containing 1% detergent NP-40 were used to determine spontaneous and maximum LDH release respectively.

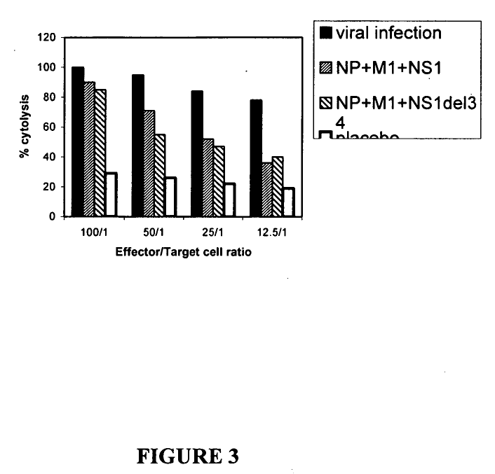

The results of CTL measurement are shown in FIG. 3. This figure shows significant CTL responses to influenza virus developed in mice vaccinated three times with DNA vaccines bearing conserved influenza NP, M1 and NS1 genes. At the E/T ratio of 25:1-50:1, CTL response reached 70-80% of target cell lysis and was similar to CTL activity developed in native infection control (mice twice inoculated with influenza A/Aichi/2/68 virus). It is seen that CTL response in mice vaccinated with the triple mixture containing pNS1del34 in addition to pNP and pM1, was slightly lower than in mice vaccinated with plasmid expressing wild-type conserved influenza proteins NP, M1 and NS1. Most likely, this could be explained by the relatively lower expression efficiency of NS1del34 protein compared to wild type NS1 (see FIGS. 1, 2). Notably, anti-influenza CTL response in naturally infected mice is known to develop mainly against NP, M1 and NS1. Therefore, it was shown that pCAGGS plasmids bearing influenza genes NP, M1, and NS1 are efficiently expressed in vivo and that three injections of these plasmid DNAs induced high CTL response against influenza virus.

Humoral anti-viral response. The level of anti-NP and M1 antibodies was determined in the sera of vaccinated mice. Serum samples of DNA-vaccinated mice were collected on day 10 following the third DNA vaccination. Sera were assayed in a direct ELISA test using whole disrupted influenza virus A/PR/8/34 adsorbed onto an ELISA plate as a target. A/PR/8/34 influenza virus was grown in chicken eggs and disrupted with non-ionic detergent to expose internal proteins NP and M1. Two-fold dilutions of animal sera were added to the pre-absorbed plates and virus-specific antibodies were measured employing anti-mouse IgG-HRP conjugate using TMB substrate.

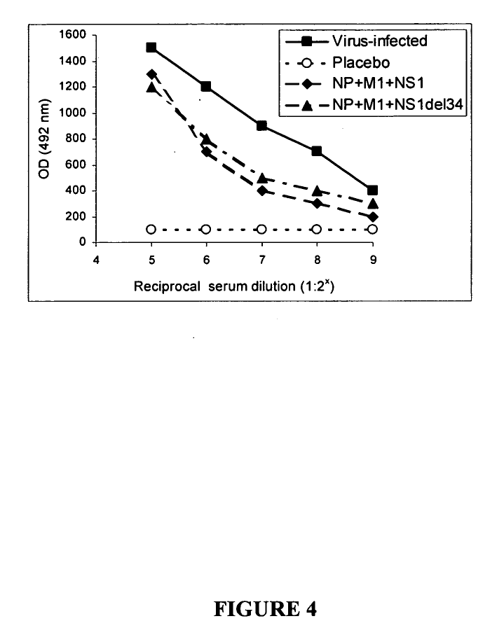

Animal antibody titers against whole influenza are shown in FIG. 4. It is clearly seen that mice infected with influenza virus (positive control) produce a prominent signal at serum dilutions as high as 1:128-1:256. A marked signal was also detected in both groups of mice vaccinated with pNP/pM1/pNS1 plasmid mixtures at dilutions of 1:64-1:128. These results lend further support to the CTL data showing effective in vivo expression of the recombinant DNA constructs encoding conserved influenza genes NP and M1, which in turn resulted in specific antibody generation. Obviously, the addition of NS1 does not increase the generation of antibodies against virus preparation since the latter does not contain NS1. Importantly, we have not seen any detrimental effect of wild-type NS1 on antibody generation, since: a) the level of antibody reactivity against virion observed in this study is similar to the one that we and other investigators have seen earlier using NP and M1 immunization only (Chen et al., Vaccine (1998), 16:1544-1549); b) lower-expressed NS1del34 did not affect anti-virion antibody generation compared to wild-type NS1. Collectively, triple combinations of conserved influenza proteins were shown to induce both cellular and humoral immune response in vivo to a level comparable to one seen in experimental infection with live virus.

Vaccines Containing Combinations of Influenza a Proteins (NP, M1 and NS1) for Protection Against Influenza Infection

The present invention provides multiple combination of influenza proteins (NP, M1, NS1) is the most efficacious in protection experiments, which are validated using one or more clinically relevant animal models such as the murine model described above. A vaccine may contain two modified NS-1 proteins, along with NP and M1. In certain embodiments, two influenza proteins are used, such as NS-1 and M1, NP and M1, or NS-1 and NP.

### Example 2

### Protective Effect Against H3N2 Influenza Virus in Experimentally Infected Mice

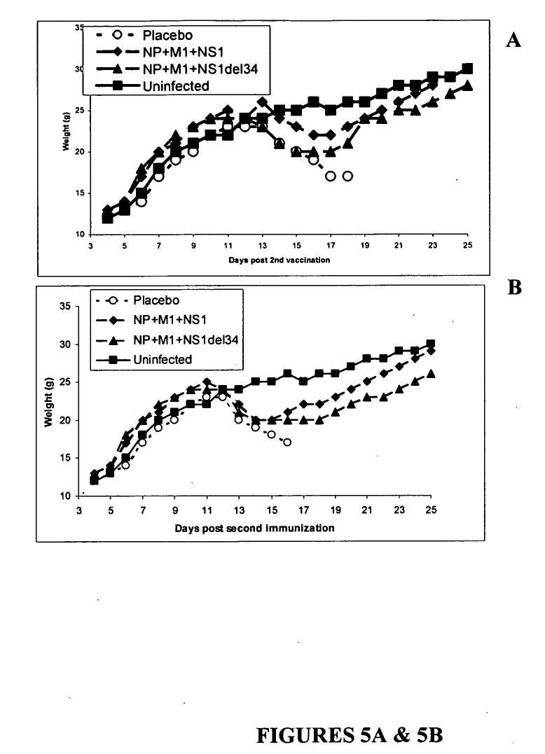

Mice vaccinated twice with both combinations of NP, M1 and NS1 (differing only in the type of NS1 used) and those in the control groups were subjected to the experimental infection with influenza virus. All animals were challenged intranasally with the mouse-adapted variant of strain A/Aichi/2/68 (H3N2) at 10 or 100 LD₅₀. Body weight, lung pathology and overall mortality were assessed. Body weight gain of mice was monitored throughout the period of observation to evaluate (i) toxicity of injected DNA samples and (ii) severity of the infection process (FIG. 5). Normal body gain was observed up to after 2^{nd} vaccination and preceding the virus infection. This data indicates the absence of any visible toxicity of vaccine DNA injections.

Immediately upon viral infection, a marked body weight reduction was observed in all infected groups. This reduction was fatal in placebo-immunized animals at both 10 and 100 LD₅₀ (FIGS. 5A, B). At the same time it was less dramatic in DNA vaccinated groups. The weight reduction in these groups was slower and body weight started to increase 3-4 days after virus infection (FIG. 5) indicating animal recovery. Importantly, in both experimental settings the body weight gain started earlier and developed more rapidly in mice vaccinated with DNA plasmids encoding wild-type proteins than in the vaccinated group where pNS1del34 was employed.

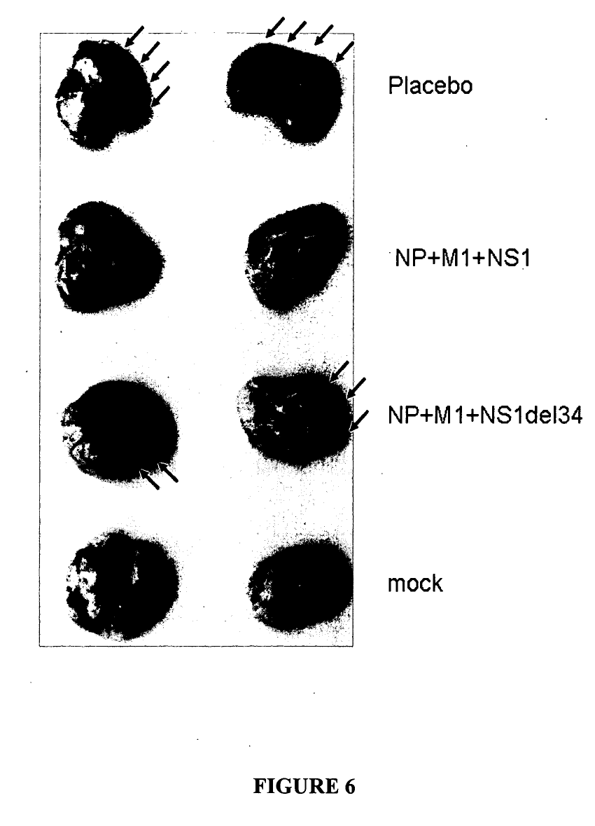

Examination of mouse lungs was done on day 6 following viral infection in the group that was infected with 10 LD₅₀. At this time, lung pathology is known to reach significant levels (Chen et al. Amer J Pathol 2003; 163:1341-1355). Two mice from each experimental group—non-vaccinated (placebo), triple-vaccinated using either pNS1 wt or pNS1del34 (in addition to pNP/pM1) and uninfected—were sacrificed, their lungs taken and photographed. Lungs of unvaccinated mice had clear signs of fatal hemorrhagic inflammation. The inflammation in DNA-vaccinated mouse lungs was significantly less than in the lungs from the placebo control group. The most significant reduction in lung pathology was observed in mice from the group vaccinated with a combination of wild-type NP, M1 and NS1 plasmids. The external appearance of lungs from this group was similar to those of mock-infected animals (FIG. 6).

Full results of animal survival following the challenge with H3N2 Aichi strain are presented in Table E1. DNA immunization.with the plasmid combination of wild-type NP, M1 and NS1 proteins resulted in a complete protection in the animals infected with a 10 LD₅₀ and showed some protective effect even when 100 LD₅₀ were used. Significantly less protection was provided by vaccination using a combination of NP, M1 and NS1del34. The survival difference between pNP/pM1/pNS1 and pNP/pM1/pNS1del34-immunized groups was statistically significant (p<0.05) for a viral challenge with 10 LD₅₀.

### Example 3

### Protective Effect Against H5N2 Influenza Virus in Experimentally Infected Mice

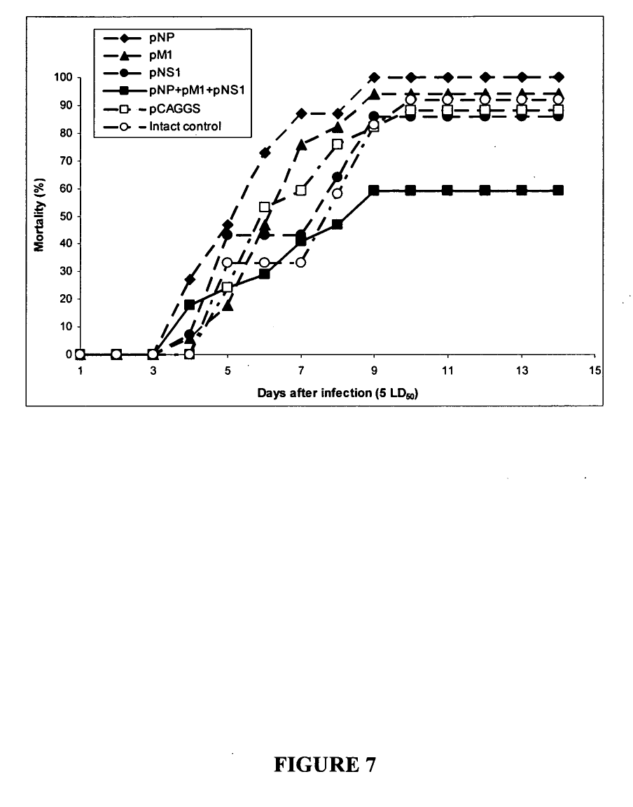

A separate experiment was performed in the mouse model using a similar scheme of immunization (as described in example 2) followed by challenge with a different influenza virus strain, A/Mallard/Pennsylvania/10218/84 (H5N2, of avian origin, but mouse-adapted). Six groups of Balb/c mice were inoculated either with a pNP/pM1/pNS1 combination or with each of plasmids separately. Vaccination was performed twice and was followed by the viral challenge with 5 LD₅₀. The data of animal survival is presented in FIG. 7 and Table E2. The only group of animals that showed a noticeable and statistically significant protection against 5 LD₅₀ H5N2 challenge was immunized by the pNP/pM1/pNS1 combination. This observation was further supported with the data on viral titer from the infected animals (Table 3). While pNP-immunized animals also showed a decrease in viral titer, it was most profoundly manifested in the group immunized by the three-plasmid combination.

### Example 4

### Immunization and Protective Effect Against H5N3 Influenza Virus in Experimentally Infected Chickens

Then the effects of immunization with the combination of pNP/pM1/pNS1 were tested in the avian model, employing another antigenically unrelated viral strain (H5N3) for the challenge. It was also imperative to test in a straight-forward manner if the addition of wild-type NS1 to pNP/pM1 combination provides an additional beneficial effect in vivo. Thus, a vaccination and experimental challenge experiment in the avian model was conducted using immunization either with pNP/pM1 or with pNP/pM1/pNS1.

Following the determination of the lethal infectious dose in the chicken model the protective effect of DNA vaccination with these plasmid combinations was assessed upon challenge with influenza H5N3 A/Tern/SA/61 virus. Viral titers in the infected birds were measured and their survival determined. Virus was not detected in the cloaks of chickens vaccinated with both pNP/pM1 and pNP/pM1/pNS1 DNA combinations.

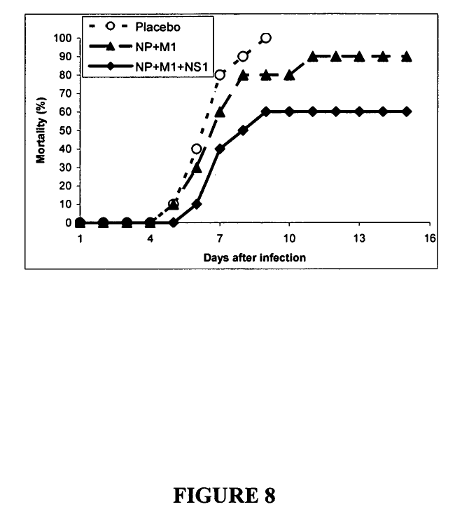

The data documenting the survival of infected chickens is presented in FIG. 8. All birds vaccinated with an empty vector (placebo) died by day 8 following challenge. Marginal protection (10-20%) was observed in the group of chickens that were vaccinated with pNP/pM1 and a more prominent protective effect (40%) was observed in the group that was vaccinated with pNP/pM1/pNS1 combination. In addition to mortality decrease, vaccination with pNP/pM1/pNS1 appeared to delay the fatal disease (FIG. 8). Birds in this group died 1-3 days later than in the placebo group. No such effect was observed in pNP/pM1-vaccinated group.

TABLE E1
Protective efficacy of DNA immunization with conserved proteins of
influenza against experimental infection with A/Aichi2/68 (H3N2)
virus in mice. The results are shown as the number of fatal infections
divided by the total number of immunized mice (10/group)

| | Lethal outcome of influenza virus | |
|---|---|---|
| | experimental infection in mice | |
| Immunizing plasmid(s) | 10 LD₅₀ | 100 LD₅₀ |
| NP + M1 + NS1 | 0/10 (100% protection) | 6/10 (40% protection) |
| NP + M1 + NS1del34 | 4/10 (60% protection) | 8/10 (20% protection) |
| Placebo | 10/10 (no protection) | 10/10 (no protection) |

TABLE E2
Survival of mice vaccinated with pNP, pM1 and pNS1 singly or in
combination after H5N2 virus A/Mallard/Pennsylvania/10218/84
infection. Numerator - number of the dead mice, denominator -
number of mice per group. Survival as of 16 days post-infection is shown.

Lethal outcome after
infection with
influenza virus (LD₅₀)

| Animals immunized with | 5 | 0.5 |
|---|---|---|
| pNP | 15/15 (100%) | Nd |
| pNS1 | 12/14 (86%) | Nd |
| pM1 | 16/17 (94%) | Nd |
| pNP/pNS1/pM1 | 10/17 (59 ± 12%) | Nd |
| pCAGGS | 15/17 (88 ± 8%) | Nd |
| Intact | 10/12 (92 ± 8%) | 2/4 |

TABLE E3
Titers of influenza virus in lungs of mice on day 4
after infection with 5 LD₅₀ of A/Mallard/Pennsylvania/10218/84.
Titration was done in MDCK cells (6 wells/dilution).

| Animals | Animals | Geometric mean titer ± SE |
|---|---|---|
| immunized with | tested | (lgTCID50/lung) |
| pNP | 4 | 5.93 ± 0.13 |
| pM1 | 4 | 6.6 ± 018 |
| pNS1 | 4 | 6.6 ± 0.18 |
| pNP/pNS1/pM1 | 4 | 5.78 ± 0.16 |
| pCAGGS | 4 | 6.3 ± 0.18 |
| Intact | 4 | 6.2 ± 0.28 |

### Example 5

### Generation of Novel DNA Constructs Expressing Immunogenic Influenza A NS1 Mutants With Decreased Interference of Host Immune System, and Vaccines Containing the Novel Constructs.

It has been shown that immunization with vectors expressing NS1 proteins provides protective benefit against influenza infection. However, the wild-type form of this protein is capable of interfering with the host immune system. Therefore, expression-competent, forms of NS1 influenza protein are generated with the regions known to be responsible for its immune modulation functions specifically mutated or deleted, and their immunogenicity and protective capacity are assessed in a clinically relevant animal model.

Plasmids expressing truncated and site-specifically changed mutants comprising the full sequence of influenza NS1 protein are constructed that do not have marked reduction in expression levels, as it has been shown above that a short deletion in the RNA binding domain dramatically decreased the expression of the NS1del34 recombinant protein and an additional deletion completely abolished the detectable expression of the resulting construct. A similar phenomenon was recently observed by other investigators using mutant NS1 forms of the related equine influenza virus (Quinlivan et al. (2005) J. Virol. 79:8431-8439). Without wishing to be bound by theory, this result may be due to the deletion of domains important to the stability of NS1 mRNA, which in its wild-type form is capable of alternative splicing and thus may be prone to degradation if changed in an adverse manner. Therefore, the present invention provides modified NS1 polypeptides that do not have significantly reduced expression levels. In one embodiment, modified NS-1 proteins are operably linked to the highly expressed marker protein, GFP, providing for determination of the expression of the mutant NS1 and its detection in vitro.

In some embodiments, the modified NS-1 protein contains a modification that is efficient, stable and is unlikely to revert directly or via compensation of function. At the same time, the vaccine vectors that are employed (e.g., DNA plasmids, vaccinia virus or adenovirus) do not present an entity that may easily and expeditiously mutate in the vaccinated subject.

There are three separate regions of the NS1 protein: the RNA binding domain (comprising amino acids 19-38, it also overlaps with nuclear localization sequence (NLS) 1, located in amino acids 34-38); the effector domain (amino acids 134-161) and the NLS2 signal (amino acids 216-221) (See, Jameson et al. (1998) J. Virology 72:8682-8689). In addition to wild-type NS1 protein, which will be used in this study as a benchmark standard, we plan to construct the following NS1 mutants and to test their expression level in vitro.

Exemplary mutants include the NS1del34 mutant (bearing 34-41 deletion, in influenza virus A this sequence is: DRLRRDQK), as well as NS1del34-38, and NS1del39-41, which have five and three amino acids of NLS 1 (part of RNA binding domain) deleted, respectively. Also generated is a modified NS-1 protein having a mutation in which the Arg-Arg sequence in positions 37-38 is changed to Ala-Ala.

Furthermore encompassed are deletion mutants that result in truncations of the C-terminus. For example, NS1mut1-99 (containing amino acids 1-99 of the NS-1 protein) and NS1mut1-125 (containing amino acids 1-125 of the NS-1 protein). Both of these mutants lack effector domain and NLS 2 sequence. Also included are mutants bearing the central and/or C-terminal domains ofNS-1. For example, NS1mut74-216 (here, the RNA binding domain and NLS 2 have been deleted), NS1mut74-237 (the RNA binding domain is deleted) and NS1mut141-237 (the RNA binding domain and a portion of the effector domain are deleted).

Nucleic acids encoding modified NS-1 forms are cloned either directly into the pCAGGS vector or via additional recloning of modified NS-1 forms into plasmid vector pd1EGFP, which bears the marker enhanced green fluorescent protein (EGFP) gene. The modified NS-1 mutants are cloned in-frame following the EGFP gene sequence, thereby creating fusion genes that will are easily detectable immunologically and are expressed efficiently. NS1-encoding plasmids are amplified and, optionally, purified by, e.g., ion exchange chromatography columns (Qiagen Endotoxin-Free). Balb/c strain mice (6-8 weeks old) are generally used for immunization experiments. Animals are vaccinated with a total of 25-100 μg of DNA dissolved in endotoxin-free PBS injected into sites in the quadriceps muscle (12.5-25 μg/leg). Two or three immunizations are performed with a 2-week period between immunizations. To determine the level of anti-NS-1 antibodies, mouse sera are taken before all immunizations and 7/14 days after the final immunization via tail bleeds, and the level of anti-NS1 antibodies will be determined in individual sera by ELISA.

Certain assays employ regents that require the presence of certain epitopes of the NS-1 protein. In certain modified NS-1 proteins, epitopes located at amino acids 34-42 (DRLRRDQKS) or 122-130 (AIMDKNIIL), are absent or present in a mutated form, which may necessitate the use of a corresponding mutated epitope peptide (for example, DLRAADQKS) for the CTL stimulation. Generally, test splenocytes are cultured with human rIL,-2 and subjected to a colorimetric CTL assay using peptide-loaded P815 mastocytoma or EL-4 target cells respectively. Non-specific lysis is measured in target cells loaded with irrelevant K^{d} or D^{b}-binding peptides.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A vaccine comprising:
a) a first nucleic acid sequence encoding an influenza nucleoprotein;
b) a second nucleic acid sequence encoding an influenza M1 protein; and
c) a third nucleic acid sequence encoding an influenza NS-1 protein,
wherein the vaccine induces a protective immune response in a mammal.

2. The vaccine of claim 1, wherein the influenza nucleoprotein comprises a modified influenza nucleoprotein.

3. The vaccine of claim 2, wherein at least one amino acid residue in the encoded protein has been substituted by one or more amino acids or at least one residue has been deleted.

4. The vaccine of claim 1, wherein the influenza M1 protein comprises a modified influenza M1 protein wherein at least one residue has been substituted by one or more amino acids or at least one residue has been deleted.

5. The vaccine of claim 1, wherein the influenza NS-1 protein comprises a modified influenza NS-1 protein wherein at least one residue has been substituted by one or more amino acids or at least one residue has been deleted.

6. The vaccine of claim 1, wherein the first, second and third nucleic acid sequences are in nucleic acid vectors.

7. The vaccine of claim 6, wherein the nucleic acid vector is selected from the group consisting of a plasmid and a viral vector.

8. The vaccine of claim 7, wherein the viral vector is selected from the group consisting of a vaccinia virus vector, adeno-associated, VEEV, Sendai-based, NDV-based and an adenovirus vector.

9. A vaccine comprising:
a) a nucleic acid sequence encoding a modified influenza nucleoprotein wherein at least one residue has been substituted by one or more amino acids or deleted.
b) a nucleic acid sequence encoding a modified influenza M1 protein wherein at least one residue has been substituted by one or more amino acids or deleted; and
c) a nucleic acid sequence encoding a modified influenza NS-1 protein wherein at least one residue has been substituted by one or more amino acids or deleted,
wherein the vaccine induces a protective immune response in a mammal.

10. The vaccine of claim 9, wherein at least one of the modified influenza nucleoprotein is more susceptible to proteolysis as compared to the polypeptide of SEQ ID NO: X1, the influenza M1 protein is more susceptible to proteolysis as compared to the polypeptide of SEQ ID NO: X2, and the influenza NS-1 protein is more susceptible to proteolysis as compared to the polypeptide of SEQ ID NO: X3.

11. The vaccine of claim 9, wherein the modified influenza NS-1 protein has an amino acid sequence of the polypeptide of SEQ ID NO: X3 or a conservative substitution thereof, wherein at least one residue has been deleted, and wherein the NS-1 protein has decreased interferon inhibitory activity as compared to the polypeptide of SEQ ID NO: X3.

12. The vaccine of claim 9, wherein the nucleic acid encoding a modified influenza nucleoprotein is present in a nucleic acid vector, wherein the nucleic acid encoding a modified influenza M1 protein is present in a nucleic acid vector, and wherein the nucleic acid encoding a modified influenza NS-1 protein is present in a nucleic acid vector.

13. The vaccine of claim 9, comprising a pharmaceutically-acceptable carrier.

14. The vaccine of claim 9, wherein the modified influenza nucleoprotein comprises an amino acid sequence selected from the group consisting of SEQ ID NO: XNP 1-10.

15. The vaccine of claim 9, wherein the modified influenza M1 protein comprises an amino acid sequence selected from the group consisting of SEQ ID NO: XM1 1-10.

16. The vaccine of claim 9, wherein the modified influenza NS-1 protein comprises an amino acid sequence selected from the group consisting of SEQ ID NO: XNS-1 1-10.

17. A vaccine comprising:
a) a nucleic acid sequence encoding an influenza nucleoprotein;
b) a nucleic acid sequence encoding an influenza M1 protein; and
c) a nucleic acid sequence encoding an influenza NS-1 protein, wherein the influenza NS-1 protein has an amino acid sequence of the polypeptide of SEQ ID NO: X3 or a conservative substitution thereof, and wherein the NS-1 protein has decreased interferon stimulatory activity as compared to the polypeptide of SEQ ID NO: X3, and wherein the vaccine induces a protective immune response in a mammal.

18. The vaccine of claim 17, wherein the influenza nucleoprotein is a modified influenza nucleoprotein comprising an amino acid sequence of the polypeptide of SEQ ID NO: X1 or a conservative substitution thereof, wherein at least one residue has been substituted by one or more amino acids or deleted, and wherein the modified influenza nucleoprotein is more susceptible to proteolysis as compared to the polypeptide of SEQ ID NO: X1.

19. The vaccine of claim 17, wherein the influenza M1 protein is a modified influenza M1 protein comprising an amino acid sequence of the polypeptide of SEQ ID NO: X2 or a conservative substitution thereof, wherein at least one residue has been substituted by one or more amino acids or deleted, and wherein the modified influenza M1 protein is more susceptible to proteolysis as compared to the polypeptide of SEQ ID NO: X2.

20. The vaccine of claim 17, where the nucleic acid sequences are contained in a single vector.

21. The vaccine of claim 17, where the nucleic acid sequences are contained in more than one vector.

22. A vaccine comprising:
a) a nucleic acid vector comprising a nucleic acid sequence encoding an influenza nucleoprotein;
b) a nucleic acid vector comprising a nucleic acid sequence encoding an influenza M1 protein; and
c) a nucleic acid vector comprising a nucleic acid sequence encoding an influenza NS-1 protein, wherein the influenza NS-1 protein has an amino acid sequence of the polypeptide of SEQ ID NO: X3 or a conservative substitution thereof, wherein at least one residue has been deleted, wherein the NS-1 protein has decreased interferon inhibitory activity as compared to the polypeptide of SEQ ID NO: X3.

23. The vaccine of claim 22, formulated to be suitable for oral, intranasal or intramuscular administration.

24. The vaccine of claim 22, wherein the nucleic acid vector is selected from the group consisting of a plasmid vector and a viral vector.

25. The vaccine of claim 24, wherein the viral vector is selected from the group consisting of vaccinia virus vector, adeno-associated, VEEV, Sendai-based, NDV-based and an adenovirus vector.

26. A vaccine comprising an isolated influenza nucleoprotein, an isolated influenza M1 protein, and an isolated influenza NS-1 protein.

27. The vaccine of claim 26, wherein the influenza nucleoprotein has an amino acid sequence of the polypeptide of SEQ ID NO: X1 or a conservative substitution thereof, wherein at least one residue has been substituted by one or more amino acids or deleted.

28. The vaccine of claim 26, wherein the influenza nucleoprotein is more susceptible to proteolysis as compared to the polypeptide of SEQ ID NO: X1.

29. The vaccine of claim 26, wherein the influenza M1 protein has an amino acid sequence of the polypeptide of SEQ ID NO: X2 or a conservative substitution thereof, wherein at least one residue has been substituted by one or more amino acids or deleted.

30. The vaccine of claim 26, wherein the influenza M1 protein is more susceptible to proteolysis as compared to the polypeptide of SEQ ID NO: X2.

31. The vaccine of claim 26, wherein the influenza NS-1 protein has an amino acid sequence of the polypeptide of SEQ ID NO: X3 or a conservative substitution thereof, wherein at least one residue has been substituted by one or more amino acids or deleted.

32. The vaccine of claim 31, wherein the influenza NS-1 protein is more susceptible to proteolysis as compared to the polypeptide of SEQ ID NO: X3.

33. The vaccine of claim 26, wherein the influenza NS-1 protein has an amino acid sequence of the polypeptide of SEQ ID NO: X3 or a conservative substitution thereof, wherein at least one residue has been deleted, wherein the NS-1 protein has decreased interferon inhibitory activity as compared to the polypeptide of SEQ ID NO: X3.

34. A vaccine comprising an isolated influenza nucleoprotein, an isolated influenza M1 protein, and an isolated influenza NS-1 protein, wherein the influenza NS-1 protein has an amino acid sequence of the polypeptide of SEQ ID NO: X3 or a conservative substitution thereof, wherein at least one residue has been deleted, wherein the NS-1 protein has decreased interferon inhibitory activity as compared to the polypeptide of SEQ ID NO: X3.

35. The vaccine of claim 34, formulated to be suitable for oral, intranasal or intramuscular administration.

36. An attenuated influenza virus comprising an NS-1 protein having an amino acid sequence of the polypeptide of SEQ ID NO: X3 or a conservative substitution thereof, wherein at least one residue been deleted, wherein the NS-1 protein has decreased interferon inhibitory activity as compared to the polypeptide of SEQ ID NO: X3.

37. A method for inducing a protective immune response against an influenza virus in a subject, comprising administering to the subject the vaccine of claim 1.

38. A method for inducing a protective immune response against an influenza virus in a subject, comprising administering to the subject the vaccine of claim 22.

39. A method for inducing a protective immune response against an influenza virus in a subject, comprising administering to the subject the vaccine of claim 26.

40. A method of formulating a vaccine, comprising combining a pharmaceutically acceptable carrier and:
a) a nucleic acid sequence encoding an influenza nucleoprotein;
b) a nucleic acid sequence encoding an influenza M1 protein; and
c) a nucleic acid sequence encoding an influenza NS-1 protein.

41. A method of formulating a vaccine, comprising combining a pharmaceutically acceptable carrier and an attenuated influenza virus comprising an NS-1 protein having an amino acid sequence of the polypeptide of SEQ ID NO: X3 or a conservative substitution thereof, wherein at least one residue has been deleted, wherein the NS-1 protein has decreased interferon inhibitory activity as compared to the polypeptide of SEQ ID NO: X3.

42. A method of formulating a vaccine, comprising combining a pharmaceutically acceptable carrier and an isolated influenza nucleoprotein, an isolated influenza M1 protein, and an isolated influenza NS-1 protein, wherein the influenza NS-1 protein has an amino acid sequence of the polypeptide of SEQ ID NO: X3 or a conservative substitution thereof, wherein at least one residue has been deleted, wherein the NS-1 protein has decreased interferon inhibitory activity as compared to the polypeptide of SEQ ID NO: X3.

43. A vaccine comprising:
a) an immunogenic peptide derived from an influenza nucleoprotein;
b) an immunogenic peptide derived from an influenza M1 protein; and
c) an immunogenic peptide derived from an influenza NS-1 protein,
wherein the vaccine induces a protective immune response in a mammal.

44. The vaccine of claim 43, wherein the immunogenic peptide derived from an influenza nucleoprotein is selected from the group consisting of CTELKLSDY, RRSGAAGAAVK, EDLTFLARSAL, ILRGSVAHK, ELRSRYWAI and SRYWAIRTR or any combination thereof.

45. The vaccine of claim 43, wherein the immunogenic peptide derived from an influenza M1 protein is selected from the group consisting of SGPLKAEIAQRLEDV, GILGFVFTL, ASCMGLIY, and any combination thereof.

46. The vaccine of claim 43, wherein the immunogenic peptide derived from an influenza NS-1 is selected from the group consisting of DRLRRDQKS and AIMDKNIIL or a combination thereof.


SEQUENCE LISTING (45 sequences)

SEQ ID NO: 1 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 2 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMVTTTNPLIRHENRMVLASTTAKAMEQMAGSSEQAAEAMEVASQARQMVQAMRTIGTHPSSSAGLKDDLLENLQAYQKRMGVQMQRFK

SEQ ID NO: 3 - protein, 230 aa, Influenza A virus
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 4 - DNA, 1497 bp, Influenza A virus
atggcgtcccaaggcaccaaacggtcttatgaacagatggaaactgatggggatcgccagaatgcaactgagattagggcatccgtcgggaagatgattgatggaattgggcgattctacatccaaatgtgcactgaacttaaactcagtgattatgaagggcggttgatccagaacagcttgacaatagagaaaatggtgctctctgcttttgatgagagaaggaatagatatctggaagaacaccccagcgcggggaaagatcctaagaaaactggagggcccatatacaagagagtagatggaagatggatgagggaactcgtcctttatgacaaagaagaaataaggcgaatctggcgacaagccaacaatggtgaggatgcgacagctggtctaactcacatgatgatctggcattccaatttgaatgatacaacataccagaggacaagagctcttgttcgcaccggaatggatcccagaatgtgctctctgatgcagggctcgactctccctagaaggtctggagctgcaggtgctgcagtcaaaggaatcgggacaatggtgatggagctgatcagaatggtcaaacgggggatcaacgatcgaaatttctggagaggtgagaatgggcggaaaacaaggagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattaatattgagagggtcagttgctcacaaatcttgcctacctgcctgtgtgtatggacctgcagtatccagtgggtacgacttcgaaaaagagggatattccttggtgggaatagaccctttcaaactacttcaaaatagccaagtatacagcctaatcagaccgaacgagaatccagcacacaagagtcagctggtatggatggcatgccattctgctgcatttgaagatttaagattgttaagcttcatcagagggaccaaagtatctccgcgggggaaactttcaactagaggagtacaaattgcttcaaatgagaacatggataatatgggatcaagtactcttgaactgagaagcgggtactgggccataaggaccaggagtggaggaaacactaatcaacagagggcctccgcaggccaaatcagtgtgcaacctacgttttctgtacaaagaaacctcccatttgaaaagtcaaccgtcatggcagcattcactggaaatacggagggaagaacctcagacatgagggcagaaatcataagaatgatggaaggtgcaaaaccagaagaagtgtctttccgtgggcggggagttttcgagctctcagacgagaaggcaacgaacccgatcgtgccctcttttgacatgagtaatgaaggatcttatttcttcggagacaatgcagaagagtacgacaattaa

SEQ ID NO: 5 - DNA, 986 bp, Influenza A virus
atgagtcttctaaccgaggtcgaaacgtacgttctctctatcgtcccgtcaggccccctcaaagccgagatcgcgcagagacttgaagatgtctttgctgggaagaacaccgatctcgaggcactcatggaatggctaaagacaagaccaatcctgtcacctctgactaaggggattttaggatttgtgttcacgctcaccgtgcccagtgagcgaggactgcagcgtagacgctttgtccagaatgcccttaatgggaatggggatccaaacaacatggacagggcagtgaaactgtacaggaagctcaaaagggaaattacattccacggggccaaagaagtagcgctcagttattctactggtgcacttgccagctgcatgggcctcatatacaacagaatggggactgtaaccactgaagtggcatttggcctagtgtgtgccacttgtgagcagattgccgactcccagcatcggtcccacagacagatggtgacgacaaccaacccactaatcagacatgagaacaggatggtgctggccagtaccacggctaaggccatggagcagatggcagggtcgagtgaacaggcagcagaagccatggaggttgctagtcaggctaggcagatggtgcaggcaatgagaaccattgggactcaccctagctccagtgccggtctaaaagatgatcttcttgaaaatttgcaggcctaccagaaacggatgggagtgcaaatgcagcgattcaagtgatcctctcgttattgccgcaagcatcattgggatcttgcacttgatattgtggattcttgatcgtcttttcttcaaatgcatttatcgtcgccttaaatacggtttgaaaagagggccttctacggaaggagtgcctgagtctatgagggaagagtatcggcaggaacagcagagtgctgtggatgttgacgatagtcattttgtcaacatagagctggagtaaaaaa

SEQ ID NO: 6 - DNA, 39 bp, Artificial sequence
  Primer
ggtgatgccccattcctttccctaagaggaaggggcagc

SEQ ID NO: 7 - protein, 248 aa, Influenza B virus
MSLFGDTIAYLLSLTEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPGNYSMQVKLGTLCALCEKQASHSHRAHSRAARSSVPGVRREMQMVSAMNTAKTMNGMGKGEDVQKLAEELQSNIGVLRSLGASQKNGEGIAKDVMEVLKQSSMGNSALVKKYL

SEQ ID NO: 8 - DNA, 39 bp, Artificial sequence
  Primer
gccccttcctcttagggaaaggaatggggcatcacctag

SEQ ID NO: 9 - DNA, 41 bp, Artificial sequence
  Primer
gcagttggagtcctcatcggagataacacagttcgagtctc

SEQ ID NO: 10 - protein, 252 aa, Artificial sequence
  Modified influenza A virus
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 11 - protein, 248 aa, Artificial sequence
  Modified influenza B virus
MSLFGDTIAYLLSLIEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPENYSMQVKLGTLCALCEKQASHSHRAHSRAARSSVPGVRREMQMVSAMNTAKTMNGMGKGEDVQKLAEELQNNIGVLRSLGASQKNGEGIAKDVMEVLKQSSMGNSALVRKYL

SEQ ID NO: 12 - DNA, 41 bp, Artificial sequence
  Primer
gagactcgaactgtgttatctccgatgaggactccaactgc

SEQ ID NO: 13 - DNA, 20 bp, Artificial sequence
  Primer
atcggcttcgccgagatcag

SEQ ID NO: 14 - DNA, 20 bp, Artificial sequence
  Primer
gttatcattccattcaagtc

SEQ ID NO: 15 - DNA, 24 bp, Artificial sequence
  Primer
ctgatgaattcaaacttctgacct

SEQ ID NO: 16 - DNA, 27 bp, Artificial sequence
  Primer
accaactcgagatggatccaaacactg

SEQ ID NO: 17 - protein, 498 aa, Influenza A virus; identical to SEQ ID NO: 1

SEQ ID NO: 18 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWLACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEEYDN

SEQ ID NO: 19 - protein, 498 aa, Influenza A virus
MASQGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWMRELVLYDKEEIRRIWRQANNGDDATAGLTHMMIWHSNLNDTTYQRTRALVRTGMDPRMCSLMQGSTLPRRSGAAGAAVKGVGTMVMELIRMIKRGINDRNFWRGENGRKTRSAYERMCNILKGKFQTAAQRAMMDQVRESRNPGNAEIEDLIFLARSALILRGSVAHKSCLPACVYGPAVASGYDFEKEGYSLVGIDPFKLLQNSQVYSLIRPNENPAHKSQLVWMACNSAAFEDLRVLSFIRGTKVSPRGKLSTRGVQIASNENMDTMGSSTLELRSRYWAIRTRSGGNTNQQRASAGQISVQPAFSVQRNLPFDKPTIMAAFTGNTEGRTSDMRAEIIRMMEGAKPEEMSFQGRGVFELSDEKATNPIVPSFDMSNEGSYFFGDNAEEYDN

SEQ ID NO: 20 - protein, 498 aa, Influenza A virus
MASQGTKRSYEQMETGGERQNATEIRASVGRMVGGIGRFYIQMCTELKLSDHEGRLIQNSITIERMVLSAFDERRNKYLEEHPSAGKDPKKTGGPIYRRRDGKWMRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMSNEGSYFFGDNAEEYDN

SEQ ID NO: 21 - protein, 498 aa, Influenza A virus
MASQGTKRSYEQMETDGERQNATEIRASVGRMIGGIGRFYIQMCTELKLSDYEGRLIQNSITIERMVLSAFDERRNKYLEEHPSAGKDPKKTGGPIYRRIDGKWMRELILYDKEEIRRIWRQANNGEDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGPAVASGYDFEREGYSLVGIDPFRLLQNSQVYSLIRPNENPAHKSQLVWMACHSAAFEDLRVSSFIRGTRVVPRGKLSTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATSPIVPSFDMSNEGSYFFGDNAEEYDN

SEQ ID NO: 22 - protein, 449 aa, Artificial sequence
  Consensus sequence
MASQGTKRSYEQMETGERQNATEIRASVGMGIGRFYIQMCTELKLSDEGRLIQNSTIERMVLSAFDERRNYLEEHPSAGKDPKKTGGPIYRRGKWRELLYDKEEIRRIWRQANNGDATAGLTHMIWHSNLNDTYQRTRALVRTGMDPRMCSLMQGSTLPRRSGAAGAAVKGVGTMVMELRMIKRGINDRNFWRGENGRTRAYERMCNILKGKFQTAAQAMMDQVRESRNPGNAEEDLFLARSALILRGSVAHKSCLPACVYGAVASGYDFEEGYSLVGIDPFLLQNSQVSLIRPNENPAHKSQLVWACSAAFEDLRVSFIGTVPRGLSTRGVQIASNENMMSTLELRSRYWAIRTRSGGNTNQQRASAGQISQPFSVQRNLPFTIMAAFGNTEGRTSDMREIIRMMEAPESFQGRGVFELSDEKAPIVPSFDMNEGSYFFGDNAEEYDN

SEQ ID NO: 23 - protein, 252 aa, Influenza A virus; identical to SEQ ID NO: 2

SEQ ID NO: 24 - protein, 252 aa, Influenza A virus
MSLLTEVETYVLSIIPSGPLKAEIAQRLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATITNPLIRHENRMVLASTTAKAMEQMAGSSEQAAEAMEVANQARQMVQAMRTIGTHPNSSAGLRDNLLENLQAYQKRMGVQIQRFK

SEQ ID NO: 25 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVALGLVCATCEHIADSQHRSHRQMATTTNPLIRHENRMVLASTTAKAMEQMAGSSEQAAEAMEVASQARQMVQAMRTIGTHPSSSAGLKDNLLENLQAYQKRMGVQVQRFK

SEQ ID NO: 26 - protein, 252 aa, Influenza A virus
MSLLTEVETYVFSIIPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEGAFGLVCATCEQIADSQHRSHRQMVTTTNPLIRHENRMVLASTTAKAMEQVAGSSEQAAEAMEVASQARQMVQAMRTIGTHPSSSAGLKDDLLENLQAYQKRMGVQMQRFK

SEQ ID NO: 27 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMVTTTNPLIRHENRMVLASTTAKAMEQMAGSSEQAAEAMEVASQARHMVQAMRTIGTHPSSSAGLKDDLLENLQAYQKRMGVQMQRFK

SEQ ID NO: 28 - protein, 237 aa, Artificial sequence
  Consensus sequence
MSLLTEVETYVSIPSGPLKAEIAQRLEDVFAGKNTDLEALMEWLKTRPILSPLTKGILGFVFTLTVPSERGLQRRRFVQNALNGNGDPNNMDRAVKLYKLKREITFHGAKEVALSYSTGALASCMGLIYNRMGTVTTEAGLVCATCEIADSQHRSHRQMTTNPLIRHENRMVLASTTAKAMEQAGSSEQAAEAMEVAQARMVQAMRTIGTHPSSAGLDLLENLQAYQKRMGVQQRFK

SEQ ID NO: 29 - protein, 230 aa, Influenza A virus; identical to SEQ ID NO: 3

SEQ ID NO: 30 - protein, 230 aa, Influenza A virus
MDSNTVSSFQVDCFLWHVRKRFADQELGDAPFLDRLRRDQKSLRGRGSTLGLDIRTATREGKHIVERILEEESDEALKMTIASVPAPRYLTEMTLEEMSRDWLMLIPKQKVTGSLCIRMDQAIMDKDIILKANFSVIFNRLEALILLRAFTDEGAIVGEISPLPSLPGHTEEDVKNAIGVLIGGLEWNDNTVRVSETLQRFTWRSSDENGRSPLPPKQKRKMERTIEPEV

SEQ ID NO: 31 - protein, 230 aa, Influenza A virus
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNITLKANFSVIFDRLETLILLRAFTEEGAIVGEISPLPSLPGHTDEDVKNAIGVLIGGLEWNDNTVRVSEILQRFAWRSNNEDGRPPLPPKQKRKMARTIESEV

SEQ ID NO: 32 - protein, 230 aa, Influenza A virus
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMNKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 33 - protein, 230 aa, Influenza A virus
MDSNTVSSFQVDCFLWHVRKRFADQELGDAPFLDRLRRDQKSLRGRGSTLGLDIETATRAGKQIVEQILEEESDGALKVTVASVPTSRYLTDMTLEEMSRDWFMLMPKQKVAGSLCIKMDQAIMDKNIILKANFSVIFNRLETLILLRAFTEEGAIVGEISPLPSLPGHTDEDVKNAIGVLIGGLEWNDNTVRVSETLQRFAWRSSDENGGPSLPPKQKRKMARTVEPEV

SEQ ID NO: 34 - protein, 198 aa, Artificial sequence
  Consensus sequence
MDSNTVSSFQVDCFLWHVRKRFADQELGDAPFLDRLRRDQKSLRGRGSTLGLDITATRGKIVEILEEESDALKTASVPRYLTMTLEEMSRDWMLPKQKVGSLCIMDQAIMKILKANFSVIFRLELILLRAFTEGAIVGEISPLPSLPGHTEDVKNAIGVLIGGLEWNDNTVRVSELQRFWRSEGLPPKQKRKMRTEEV

SEQ ID NO: 35 - protein, 9 aa, Influenza virus
CTELKLSDY

SEQ ID NO: 36 - protein, 11 aa, Influenza virus
RRSGAAGAAVK

SEQ ID NO: 37 - protein, 11 aa, Influenza virus
EDLTFLARSAL

SEQ ID NO: 38 - protein, 9 aa, Influenza virus
ILRGSVAHK

SEQ ID NO: 39 - protein, 9 aa, Influenza virus
ELRSRYWAI

SEQ ID NO: 40 - protein, 9 aa, Influenza virus
SRYWAIRTR

SEQ ID NO: 41 - protein, 15 aa, Influenza virus
SGPLKAEIAQRLEDV

SEQ ID NO: 42 - protein, 9 aa, Influenza virus
GILGFVFTL

SEQ ID NO: 43 - protein, 8 aa, Influenza virus
ASCMGLIY

SEQ ID NO: 44 - protein, 9 aa, Influenza virus
DRLRRDQKS

SEQ ID NO: 45 - protein, 9 aa, Influenza virus
AIMDKNIIL